(19) United States
(12) Patent Application Publication
(10) Pub. No.: US 2013/0131486 A1
(43) Pub. Date: May 23, 2013

(54) COMPUTER PROGRAM FOR SPINE MOBILITY SIMULATION AND SPINE SIMULATION METHOD

(71) Applicants: Franz Copf, Stuttgart (DE); Norbert Welsch, Tubingen (DE); Thomas Welsch, Tubingen (DE); Eric Louvard, Metzingen (DE); Frank Hanisch, Tubingen (DE); Gerhard Pross, Weil im Schonbuch (DE); Erik Schols, Stuggart (DE)

(72) Inventors: Franz Copf, Stuttgart (DE); Norbert Welsch, Tubingen (DE); Thomas Welsch, Tubingen (DE); Eric Louvard, Metzingen (DE); Frank Hanisch, Tubingen (DE); Gerhard Pross, Weil im Schonbuch (DE); Erik Schols, Stuggart (DE)

(73) Assignee: spontech spine intelligence group AG, Stuttgart (DE)

(21) Appl. No.: 13/580,489

(22) PCT Filed: Feb. 25, 2011
(86) PCT No.: PCT/EP11/00923

§ 371 Date: Feb. 6, 2013

Related U.S. Application Data
(60) Provisional application No. 61/308,486, filed on Feb. 26, 2010.
(60) Provisional application No. 61/378,469, filed on Aug. 31, 2010.

(51) Int. Cl. A61B 5/107

(52) U.S. Cl. CPC A61B 5/1079
USPC 600/407

(57) ABSTRACT

A computer program for spine mobility simulation is configured, if running on a computer, to cause the computer to perform the following steps: a) accessing bio-metric data which relate to the spine of a patient, the spine having at least one compromised spine segment; b) displaying a model of the spine of the patient comprising a plurality of vertebrae; c) enabling a user to change the position of at least one of the vertebrae of the spine model; d) computing the effects of the position change on the remaining vertebrae; e) displaying the spine model in a new configuration, thereby taking into account the position changed by the user in step c) and the position changes of the remaining vertebrae computed in step d).

### BACKGROUND OF THE INVENTION

1. Field of the Invention

The present invention relates to a computer program for spine mobility simulation. Such a program may be used for configuring a spine implant, for example a cage or another fusion implant, or a non-fusion implant such as an intervertebral disk prosthesis. The present invention also relates to a spine simulation method.

2. Description of Related Art

In the treatment of diseases, injuries or malformations affecting spinal motion segments, and especially those affecting disk tissue, it has long been known to remove some or all of a degenerated, ruptured or otherwise failing disk. In cases involving intervertebral disk tissue that has been removed or is otherwise absent from a spinal motion segment, corrective measures are indicated to ensure the proper spacing of the vertebrae formally separated by the removed disk tissue.

Sometimes the two adjacent vertebrae are fused together using transplanted bone tissue, an artificial fusion component or other devices. Artificial fusion components are typically formed by cages such as described in US 2003/0045938 A1 or US 2007/0055374 A1.

Spinal fusion procedures, however, have raised concerns in the medical community that the bio-mechanical rigidity of intervertebral fusion may predispose neighboring spinal motion segments to rapid deterioration. More specifically, unlike a natural intervertebral disk, spinal fusion prevents the fused vertebrae from pivoting and rotating with respect to one another. Such lack of mobility tends to increase stresses on adjacent spinal motion segments.

As an alternative to fusion techniques, different types of intervertebral disks arthroplastic prostheses have been employed to prevent the collapse of the intervertebral disk compartment between adjacent vertebrae while maintaining a certain degree of stability and range of pivotal and rotational motion there between. Such devices typically include two or more particular components that are attached to respective upper and lower vertebrae. Various types of such prostheses are disclosed in US 2005/0071007, US 2005/0203626 A1, US 2004/0225362 A1, US 2006/0142862 A1 and US 2005/0234553 A1, for example.

Spine implants are available in different sizes, taking into account that the size and shape of the vertebrae significantly vary along the human spinal column. For example, implants for disk compartments in the cervical spinal column are usually much smaller than implants for disk compartments in the lumbar spinal column. With regard to fusion components in the form of cages, it has also been proposed to use cages having a tapered shape, with the tapering angle being adapted to the lordosis prevailing in the disk compartment in which the cage is implanted. Suppliers of spine implants provide the surgeons with tables in which they can look up which implant is suitable for the specific disk compartment where an implant shall be implanted.

However, it has turned out that even if prostheses allowing pivotal and rotational movements are used, the results of the implant surgery are still often unsatisfactory. The present inventors have discovered that in many cases this is due to the fact that the implants do not allow for the specific needs of the individual patient. More particularly, the conventional approaches fail to exactly restore the required mobility of the affected vertebrae, and they also ignore the natural mobility range of the adjacent vertebrae in the respective spine segment which usually differs from patient to patient.

For the fusion approach, one of the inventors has proposed to offer the surgeon not just a few different cages, but a very large number (for example up to 100) of cages that differ with respect to at least three geometric quantities, for example tapering angle, length and thickness of the cage. To make optimum use of this, the surgeon must be able to select the cage which is best suited for the specific patient and the vertebrae that shall be fused. For example, wrong tapering angles inevitably lead to more stress than necessary on adjacent joints. A wrong cage length may cause a cage to sink, at some time after the surgery, into the soft bone tissue of the vertebrae that is surrounded by a harder bone ring. If that happens, the correct angle between the vertebrae, which has been originally established by the cage, is not maintained any more.

As a matter or course, the problem of determining the optimum implant configuration also occurs if adjustable cages are used. Such adjustable cages make it possible to change certain geometric parameters, for example the tapering angle, by adjusting a setting screw, for example, and may thus help to reduce the overall number of cages that have to be stored and sterilized.

For the non-fusion approach, one of the inventors has proposed in WO 2007/003438 A2 a modular intervertebral disk prosthesis which can be individually configured so that it is perfectly adapted to the needs of a specific patient. In one embodiment this modular intervertebral disk prosthesis comprises support plates and various inserts that carry joint members, stops delimiting the range of motion, and caps formed like a dome which penetrates into the relatively soft bone tissue (substantia spongiosa) within the rigid circumferential bone ring of the vertebrae. By assembling these components from a kind of construction kit, the surgeon is able to position the center of motion at a location where he expects that the natural mobility of the spine segment is completely restored.

However, it is difficult for a surgeon to determine the optimum fusion cage or the optimum configuration of a prosthesis solely on the basis of experience and certain biometrical data which he has obtained for the relevant spine segment, for example using medical imaging techniques.

### SUMMARY OF THE INVENTION

It is therefore an object of the present invention to provide means that assist the surgeon in determining a configuration of a spine implant which is most suited for being implanted into the patient.

This object is achieved by a computer program which is configured, if running on a computer, to cause the computer to perform the following steps:
a) accessing biometric data which relate to the spine of a patient, the spine having at least one compromised spine segment;
b) displaying a model of the spine of the patient comprising a plurality of vertebrae, for example with alphanumerical symbols or graphically;
c) enabling a user to change the position of at least one of the vertebrae of the spine model;
d) computing the effects of the position change on the remaining vertebrae;
e) displaying the spine model in a new configuration, thereby taking into account the position changed by the user in step c) and the position changes of the remaining vertebrae computed in step d).

The computer program thus simulates the patient's spine, and if the user changes the position of at least one of the vertebrae, he will be provided with a forecast what the impact of this change on the remaining vertebrae will be. This significantly simplifies the choice of the optimum implant, because the surgeon is now able to simulate how different implants would affect the patient's spine.

Usually the model of the spine will not include all vertebrae of the patient, but only some of them. For example, the simulation may be restricted to the lumbar or cervical vertebrae. Consequently the remaining vertebrae, for which the effect of the position change is computed, are not all the other vertebrae of the spine, but only those vertebrae to which the simulation extends.

In one embodiment the biometric data comprise data relating to the compromised spine segment and data relating to at least one healthy spine segment which may be arranged directly adjacent to the degenerated spine segment or some distance away from it. This makes it possible to take into account a larger segment of the spine, because also the specific mobility of the vertebrae further away from the implant may have a significant impact on the optimum configuration of the implant, for example with regard to tapering angles in the case of fusion implants, or the location of the center of motion in the case of non-fusion implants.

The computer program may prompt the user to assign a first identifier to one of the vertebrae. It will then automatically recognize the vertebrae shown in the image using pattern recognition algorithms, and automatically assign different identifiers to the other displayed vertebrae. Finally the identifiers assigned before are displayed in the image. However, instead of a fully automatic recognition and labeling routine an interactive process with computer assisted labeling may be used.

The computer program may offer the user to determine the geometry of an intervertebral disk compartment, which is located within the compromised spine segment, by arranging at least four points on or in close vicinity to edges of vertebrae which are shown in the image and between which the intervertebral disk compartment is formed.

In one embodiment the computer program performs the additional step of accessing data, which have been obtained during the implant surgery as a result of measurements (automatically or performed by the surgeon), but before the implant is implanted. Such data may relate to a pressure or distraction force which is produced between the adjacent vertebrae if a certain distance is established by the later insertion of the implant. Such data measured by the surgeon during surgery may also be stored in the biometric database.

Similarly, also other biometric data relating to the spine of the patient may be added to the biometric database which is accessed by the program. Furthermore, biometric data of each patient may be added to the biometric database. This results in a self-learning effect and thus improves the accuracy of the forecast produced by the computer program.

The computer program may even provide the surgeon with one or more proposals for a specific implant configuration.

The implant may be a fusion implant which does not provide mobility of a spine segment fused by the fusion implant. Such a fusion implant may comprise a cage which is configured to be inserted into an intervertebral disk compartment. In the case of fusion implants the correct position of the vertebrae to be fused is crucial for the success of the surgery, and the computer program proposes a configuration of the implant which ensures this optimum relative position of the vertebrae. The parameters defining the fusion implant configuration may include length and the diameter at one or more axial positions.

If the implant is a non-fusion implant which maintains the mobility of adjacent vertebrae, then also the parameters related to this mobility may have to be taken into account by the computer program. Among others, the optimum non-fusion implant must have a center of motion which is exactly located where it should be. This optimum location may be the location where it has been before the degeneration has commenced. In some cases, however, the condition of the neighboring vertebrae requires that this location be shifted so as to reduce strains on the neighboring vertebrae and the surrounding ligaments and other tissues.

Subject of the invention is also a spine simulation method, comprising the steps of:
a) accessing biometric data which relate to the spine of a patient, the spine having at least one compromised spine segment;
b) displaying a model of the spine of the patient comprising a plurality of vertebrae using a computer;
c) enabling the user to change the position of at least one of the vertebrae of the spine model;
d) computing the effects of the position change on the remaining vertebrae;
e) displaying the spine model in a new configuration, thereby taking into account the position changed by the user in step c) and the position changes of the remaining vertebrae computed in step d).

The biometric data may comprise data relating to the compromised spine segment and data relating to at least one healthy spine segment which is arranged adjacent to the compromised spine segment.

The biometric data may comprise image data obtained from images of the patient's spine in different spine positions, the images having been taken by using medical imaging techniques.

The different spine positions may comprise a neutral position, a fully extended position and a fully flexed position.

The method may comprise the step of displaying one of the images of the patient's spine or a portion thereof.

The method may comprise the following additional steps:
i) assign a first identifier to one of the vertebrae;
ii) automatically recognizing the vertebrae shown in the image using pattern recognition algorithms;
iii) automatically assigning different identifiers to the other displayed vertebrae;
iv) displaying the identifiers assigned in step iii) in the image.

The method may comprise the step of assigning a degeneration parameter to at least one vertebra and/or to at least one intervertebral disk.

The method may comprise the step of taking into account patient related data, in particular age, sex, height and body mass index, during step d).

The method may comprise the step of determining the edges of the vertebrae using an edge detection algorithm.

The method may comprise the step of displaying the edges and offering the user to modify the displayed edges.

The method may comprise the step defining the geometry of an intervertebral disk compartment, which is located within the compromised spine segment, by four points on or in close vicinity to edges of vertebrae which are shown in the image and between which the intervertebral disk compartment is formed.

The method may comprise the step of displaying the four points in the image in such a manner that the four points are always located on a variable isosceles trapezoid.

The method may comprise the steps of determining the geometry of the intervertebral disk compartment based on the edges determined by using the edge detection algorithm and displaying the determined geometry in the image.

The method may comprise the step of assigning a measured reference value to a geometric parameter of one of the vertebrae that are displayed in the image.

The method may comprise the step of computing the real dimensions of the isosceles trapezoid based on the measured reference value.

The method may comprise the step of determining, for at least some of the vertebrae shown in the images, the ability to move relative to a neighboring vertebra, and to determine parameters describing this ability.

The method may comprise the step of determining pivotal angles for maximum extension and maximum flexion.

The method may comprise the step of determining a pivotal axis for pivotal movements between adjacent vertebrae.

The method may comprise determining a range of motion for at least some of the vertebrae shown in the images, wherein the range of motion is defined as the difference between the pivotal angles for maximum extension and maximum flexion of the spine.

The method may comprise the step of distributing in step d) an angle change input by the user among a plurality of adjacent vertebrae.

The method may comprise the step of distributing the angle change among the plurality of adjacent vertebrae in proportion to the range of motion determined for these vertebrae.

The method may comprise comparing the parameters relating to the patient to corresponding parameters relating to other persons and stored in a database.

The other persons may be selected by comparing degeneration parameters of the other persons to degeneration parameters of the patient.

The method may comprise the step of modifying the parameters of the patient by statistically analyzing the parameters of the other persons.

The method may comprise the step of adding the biometric data obtained for the patient to the database.

The method may comprise the step of computing the model of the patient's spine using the parameters relating to the patient.

The method may comprise the step of reading data from an implant database in which data relating to all available components of the implant are stored.

The method may comprise the step of accessing data, which have been obtained during the implant surgery as a result of measurements, but before an implant is implanted, and entering these data into the spine model.

The method may comprise the step of outputting a proposal for a treatment of the compromised spine segment.

The method may comprise the step of outputting a proposal for an implant configuration.

The implant comprises a cage which is configured to be inserted into an intervertebral disk compartment, or the implant is an intervertebral disc prosthesis which is configured to be inserted into an intervertebral disc compartment.

The method may comprise the step of determining the geometry of an intervertebral disc compartment, which is located within the compromised spine segment, by performing the following steps:
(a) accessing a database in which geometries of intervertebral disc compartments of other persons are stored;
(b) identifying other persons having intervertebral disc compartments which have a similar geometry as corresponding intervertebral disc compartments of the patient in non-compromised spine segments, wherein the similarity is determined by an algorithm;
(c) from the persons identified in step (b), using the geometry of the intervertebral disc compartment, which corresponds to the intervertebral disc compartment of the compromised segment of the patient, to compute a mean geometry;
(d) determining an implant that will, if inserted into the intervertebral disc compartment of the compromised spine segment of the patient, change its geometry such that it is at least substantially identical to the mean geometry determined in step (c).

The method may comprise the step of displaying a graph in which, for at least two adjacent vertebrae, the position of maximum inclination, the position of maximum reclination and the neutral position are shown as symbols on a horizontal scaled line.

The method may comprise of displaying statistical data obtained from other persons for at least one of the positions indicated on the scaled line by symbols.

The method may comprise the step of displaying the statistical data as frequency distribution curve.

Subject of the invention is also a method, comprising the following steps:
a) accessing biometric data which relate to the spine of a patient, the spine having at least one compromised spine segment;
b) accessing biometric data which relate to the spine of other persons;
c) comparing the biometric data accessed in step a) to the biometric data accessed in step b);
d) outputting a proposal for a configuration of an implant that is to be inserted into an intervertebral disc compartment of the compromised spine segment.

The implant may comprise a cage which is configured to be inserted into the intervertebral disk compartment, or the implant may be an intervertebral disc prosthesis which is configured to be inserted into the intervertebral disc compartment.

The method may comprise the step of determining the geometry of an intervertebral disc compartment, which is located within the compromised spine segment, by performing the following steps:
(a) accessing a database in which geometries of intervertebral disc compartments of other persons are stored;
(b) identifying other persons having intervertebral disc compartments which have a similar geometry as corresponding intervertebral disc compartments of the patient in non-compromised spine segments, wherein the similarity is determined by an algorithm;
(c) from the persons identified in step (b), using the geometry of the intervertebral disc compartment, which corresponds to the intervertebral disc compartment of the compromised segment of the patient, to compute a mean geometry;
(d) determining an implant that will, if inserted into the intervertebral disc compartment of the compromised spine segment of the patient, change its geometry such that it is at least substantially identical to the mean geometry determined in step (c).

The method may comprise the step of outputting a proposal for an instrument that should be used when inserting the implant into the intervertebral disc compartment, wherein the instrument is adapted to the configuration of the implant proposed in step d).

### BRIEF DESCRIPTION OF THE DRAWINGS

Various features and advantages of the present invention may be more readily understood with reference to the following detailed description of preferred embodiments taken in conjunction with the accompanying drawings in which:

FIG. 1 is a side view of a human spine;

FIG. 2 is a side view of two vertebrae of the spine shown in FIG. 1;

FIG. 3 is a sectional view through an intervertebral disk prostheses;

FIG. 4 is a perspective view of a sub-set of different upper supporting plates which may be used for assembling the prosthesis shown in FIG. 3;

FIG. 5 is a perspective view of a sub-set of different guiding plates which may be used for assembling the prosthesis shown in FIG. 3;

FIG. 6 is a perspective view of a sub-set of different cap inserts which may be used for assembling the prosthesis shown in FIG. 3;

FIG. 7 is a perspective view of a sub-set of different joint elements inserts which may be used for assembling the prosthesis shown in FIG. 3;

FIG. 8 schematically illustrates the main function of the computer program;

FIG. 9 schematically illustrates the main modules of the computer program according to a first embodiment which is adapted to support the surgeon in selecting a suitable prosthesis;

FIG. 10 is a flow diagram showing important steps performed by the computer program according to the first embodiment;

FIG. 11a to 11c are sagittal digital X-ray images of three adjacent vertebrae of the patient's spine;

FIG. 12 is the image of FIG. 11a, but with prepared labels for identifying the vertebrae;

FIG. 13 is the image of FIG. 11a, but with additional input field for inputting degeneration parameters;

FIG. 14 is the image of FIG. 11a after edge detection;

FIG. 15 is an illustration how the images are matches so as to determine the individual range of motion for each vertebra;

FIG. 16 is an enlarged cutout from the lower portion of FIG. 15;

FIG. 17 is a graphic illustration of the range of motion of a single vertebra;

FIG. 18 is a graphic illustration of the simulated spine of the patient;

FIG. 19 is a graphic illustration as in FIG. 18, but after the position of a single vertebra has been changed by the user;

FIG. 20 is an illustration of the motional degrees of freedom which the vertebrae have;

FIG. 21 is an illustration showing the projection of a vertebra by an X-ray source on an X-ray detector;

FIG. 22 is a perspective view on a fusion implant;

FIG. 23 is cross sectional view through six differently sized fusion implants having the same general constitution as the implant shown in FIG. 22;

FIG. 24 schematically illustrates, in a representation similar to FIG. 8, the main function of the computer program according to a second embodiment in which the computer program is adapted to support the surgeon in selecting a suitable fusion implant;

FIG. 25 is a flow diagram showing important steps performed by the computer program according to the second embodiment;

FIG. 26 is a side view on an intervertebral disc compartment that is formed between two adjacent vertebrae;

FIG. 27 is a sagittal digital X-ray image of three adjacent vertebrae of the patient's spine in which the surgeon is prompted to enter an absolute reference value for the length of the diagonal through one of the vertebrae;

FIG. 28a is a sagittal digital X-ray image of five adjacent vertebrae of the patient's spine in which a compromised and healthy intervertebral disc compartments are highlighted;

FIG. 28b is a sagittal digital X-ray image of five adjacent vertebrae of the patient's spine in which a fusion implant has been graphically inserted into the compromised intervertebral disc compartment;

FIG. 29 is an exemplary result screen which displays a proposed fusion cage as well as a sleeve that the surgeon may use to insert the proposed cage into the intervertebral disc compartment.

### DESCRIPTION OF PREFERRED EMBODIMENTS

1. Introduction

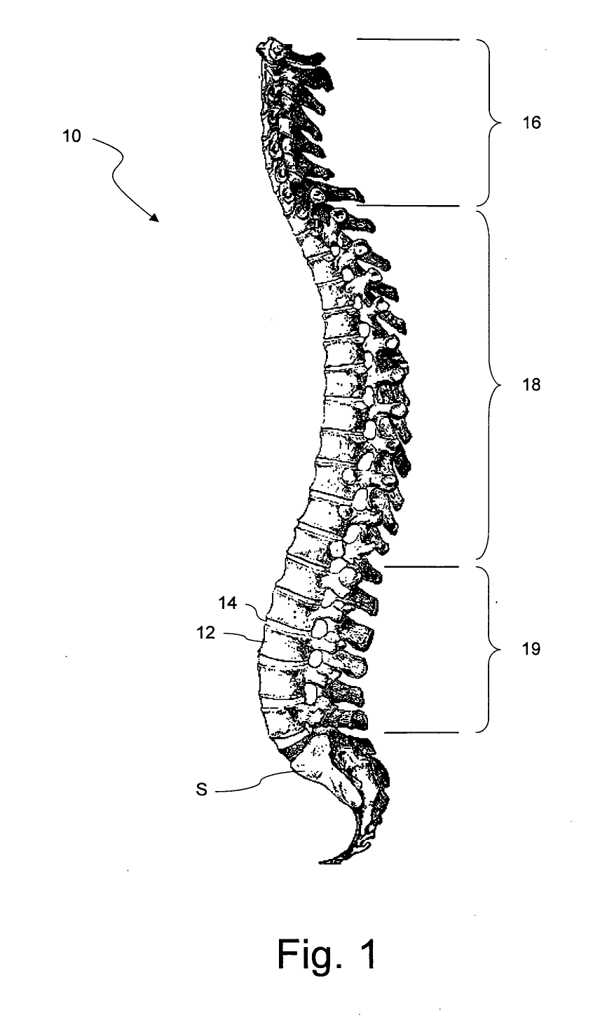

FIG. 1 is a side view of a human spine comprising vertebrae 12 and intervertebral disks 14 which are arranged in intervertebral disk compartments formed between adjacent vertebrae 12. The first seven vertebrae 12 of the spine 10, counted from its top, form the cervical spinal column 16, the following twelve vertebrae the thoracic spinal column 18, and the remaining five vertebrae 12 the lumbar spinal column 19. The latter is connected by the sacrum S to the pelvic. The cervical and lumbar spinal columns 16, 19 have a lordotic curvature, whereas the thoracic spinal column 18 has a kyphotic curvature. From FIG. 1 it becomes clear that the vertebrae 12 and also the intervertebral disks 14 have different shapes and sizes. This, in turn, results in the different curvatures and also in different mobilities of the spinal segments. Thus the joints formed by pairs of adjacent vertebrae 12 have properties that significantly differ along the spinal column.

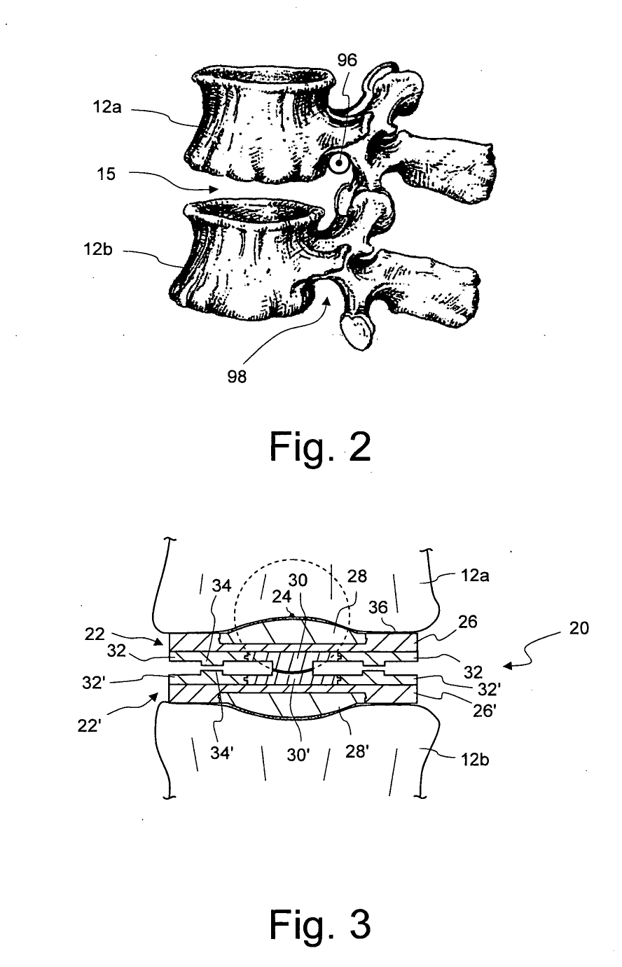

FIG. 2 is an enlarged cut-out of FIG. 1 in which two adjacent vertebrae 12a, 12b are shown, with the intervertebral disk removed so that the intervertebral disk compartment 15 is empty. If a disk 14 is degenerated so that it has to be removed by the surgeon, the intervertebral disk compartment must not remain empty, as is shown in FIG. 2. If a surgeon decides that a fusion implant shall be inserted, the two adjacent vertebrae 12a, 12b are rigidly connected by the implant such that bone material is allowed to grow between the adjacent vertebrae 12a, 12b and finally fuses them. If the surgeon decides to use a non-fusion implant, i.e. an intervertebral disk prosthesis, the implant is inserted in the intervertebral disk compartment 15 and thus replaces the removed disk 14. The prosthesis maintains, at least to a certain extent, the ability of the adjacent vertebrae to move relative to each other.

2. Modular Intervertebral Disk Prosthesis

FIG. 3 is a sectional view through an intervertebral disk prosthesis 20 as it is described in WO 2007/003438 A2 filed by one of the present inventors. The full disclosure of this earlier application is incorporated herein by reference.

The prosthesis 20 comprises an upper element 22a and a lower element 22′ that can perform pivoting movements with regard to a center of motion denoted by 24 and also rotational movements along a longitudinal axis extending along the spinal column. The upper element 22 comprises a supporting plate 26 which receives on one side a cap insert 28 and on the opposite side a joint element 30 and two guide plates 32. Each of the guide plates 32 has a projection 34 that delimits the pivoting range.

The lower element 22′ is configured in the same manner; in order to make corresponding components of the upper element 22 and the lower element 22′ distinguishable, the components of the lower element 22′ are designated with dashed reference numerals.

In the implanted state as shown in FIG. 3, the cap inserts 28, 28′, which may have a polished surface, are placed within the rigid circumferential bone ring 36 of the adjacent vertebrae 12a, 12b. The forces exerted by the vertebrae 12a, 12b on the prosthesis 20 are mainly taken by the portions of the supporting plate 26, 26′ which surround the cap inserts 28 and 28′, respectively. These portions rest on the rigid circumferential bone ring 36 of the vertebrae 12a, 12b.

As it has been mentioned above, in a healthy human spine the joints formed by pairs of adjacent vertebrae 121, 12b have properties that significantly differ along the spinal column. Therefore all or at least some of the components of the prosthesis 20 shown in FIG. 3 have to be adapted to these different properties. As a matter of course, these properties also differ significantly from one patient to the other. For example, parameters such as age, sex, height and weight of a patient have an influence on these properties. Genetically caused or acquired degenerations of the vertebrae 12 in the affected spinal segment and also in neighboring spinal segments make it difficult to accurately forecast the optimum configuration of the prosthesis 20 even if the aforementioned parameters, namely age, sex, height and weight of a patient, are known.

This particularly applies to the location of the center of motion 24 which is decisive for the extent to which the prosthesis 20 will functionally fit into the affected segment of the spinal column. For example, if the mobility of the adjacent vertebrae 12a, 12b is restricted, for example due to original or acquired degenerations of the vertebrae 12a, 12b, the center of motion 24 does not necessarily have to be located where it would be in a similar healthy spinal column segment. Instead, the reduced mobility of vertebrae further away may require certain adaptations such that these vertebrae and the surrounding ligaments and other tissues are not unduly strained. An ideal position of the center of motion 24 determined by the prosthesis 20 therefore requires a thorough analysis of the mobility of the respective spinal segment and the functional interaction between the components of this spinal segment.

Also the maximum pivoting range, which is, in the embodiment shown in FIG. 3, mainly determined by the projections 34, 34′ of the guide plates 32, 32′, has to be carefully adapted to the requirements of the specific patient.

In the prosthesis 20 shown in FIG. 3 all components are part of a modular system which comprises a plurality of sets of differently sized and/or shaped components so that the surgeon is able to assemble a prosthesis 20 which is ideally adapted to the specific needs of the patient. For example, if the pivoting range allowed by the prosthesis 20 shown in FIG. 3 shall be greater than indicated, the guide plates 32, 32′ may be replaced by different guide plates having lower projections 34, 34′. If the center of motion 24 shall be moved more to the top of the page such that it is arranged a few millimeters within the upper vertebra 12a, the joint elements 30 may be replaced by different joint elements in which the spherical surfaces have a greater radius of curvature. Other modifications are explained in more detail in the above mentioned WO 2007/003438 A2.

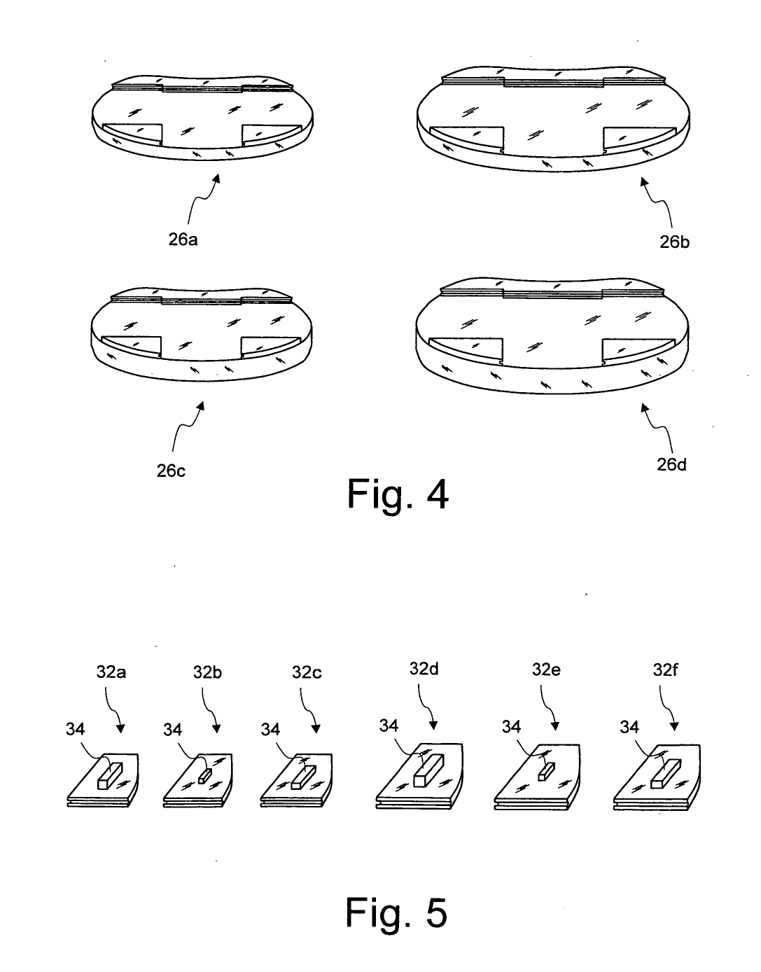

In the following some of the sets will be described with reference to FIGS. 4 to 8.

FIG. 4 shows a subset of four different upper supporting plates 26 in a perspective view and upside down. The two supporting plates 26a, 26c shown on the left hand side of FIG. 4 are generally smaller than the other two supporting plates 26b and 26d shown on the right hand side. These plates 26a, 26c may be suited for being implanted into the cervical spinal column 16, whereas the other two plates 26b, 26d may be implanted into the lumbar spinal column 19.

Within plates of identical diameter the thickness of the plates is different. More particularly, the two plates 26a, 26b shown on top have a smaller thickness than the other two plates 26c, 26d.

It is to be understood that the complete set of upper supporting plate 26 may comprise considerably more different plates. The plates may differ with respect to other dimensions, for example width or length, or they may have a wedge-like shape with different wedge angles.

FIG. 5 is a perspective view of a sub-set of guide plates 32. The three guide plates 32a, 32b and 32c shown on the left hand side of FIG. 5 are smaller than the three guide plates 32d, 32e and 32f shown on the right hand side. Apart from that, within guide plates of the same size, the shapes and sizes of the projection 34 differ which will result in different pivoting ranges.

It is to be understood that the complete set of guide plates may comprise significantly more different guide plates than shown in FIG. 5. For example, in some guide plates 32 the projection 34 may have other shapes or may be made of a resilient material.

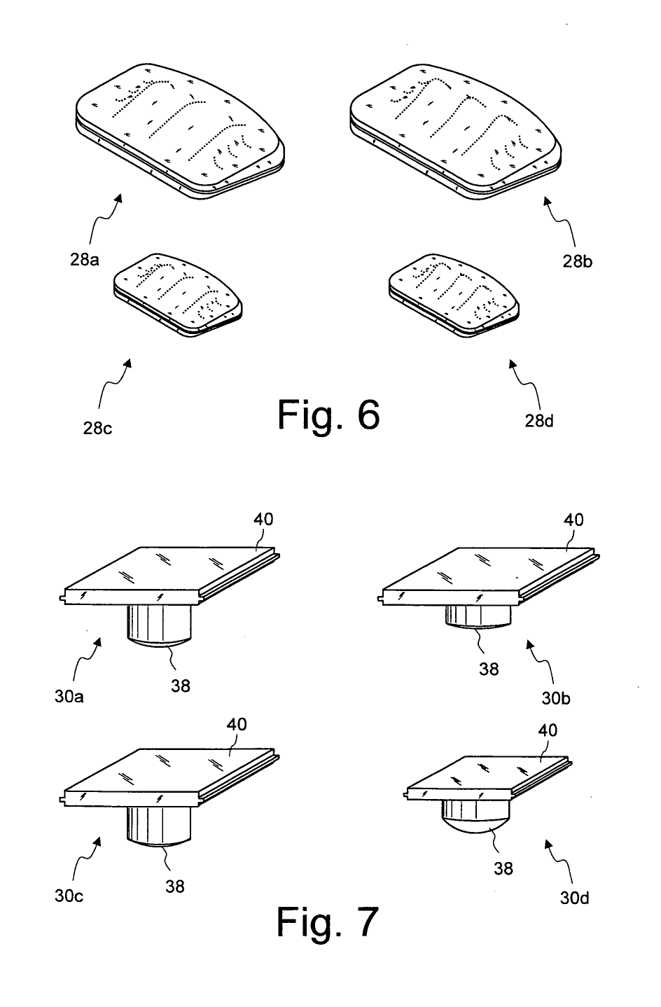

FIG. 6 is a perspective view of a sub-set of cap inserts. The general configuration of the cap inserts is described in the above mentioned WO 2007/003438 A2 and will not be explained here again. In FIG. 6 the cap inserts 28a, 28b shown on top are provided for larger supporting plates than the two other inserts 28c, 28d. Among inserts of the same overall size, the inserts 28a, 28c shown on the left hand side have a smaller height and ramp steepness than the inserts 28b and 28d.

It is to be understood that the complete set of cap inserts 28 may comprise still further different cap inserts. For example, other cap inserts may be made of different materials or may have different surface properties (for example rough or polished surfaces).

FIG. 7 is a perspective view of a sub-set of joint elements. The joint elements 30a, 30b and 30c are provided for the same supporting plate size, but differ with respect to the curvature of the spherically curved joint surfaces 38 and/or the distance of these surfaces from the opposite plane surface 40 of the joint elements 30.

It is to be understood that the complete set of joint elements 30 may comprise still further different elements. For example, some joint elements may have non-spherical joint surfaces 38, or the projections bearing the joint surfaces may be arranged in an inclined manner with regard to the insert plates on which they are fixed.

It is difficult for a surgeon to assemble the ideal prosthesis 20 merely on the basis of experience and some biometric data relating to the specific patient. In the following a computer program will be described that supports the surgeon in his task of suitably selecting the components of the prosthesis 20 so that the patient regains optimum mobility without unduly straining other portions of the spinal column that would ultimately result in new discomforts and pains. It is to be understood that in principle the same steps may be taken to determine the configuration of a fusion implant that is best suited for the degenerated intervertebral disk compartment of a particular patient (see below section 4).

2.1 Computer program

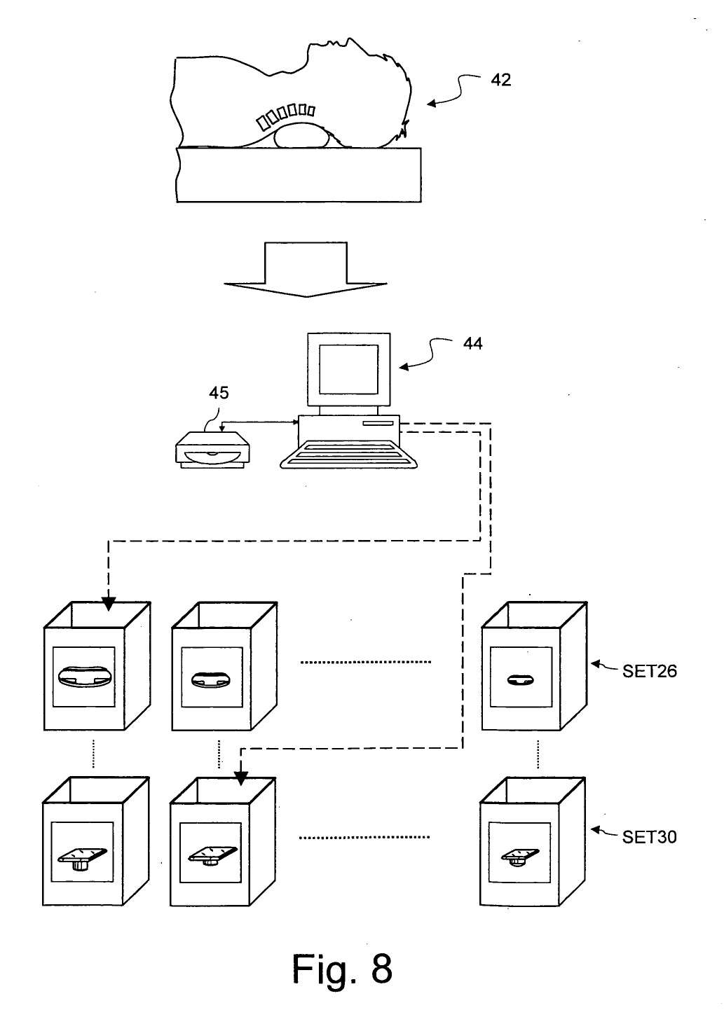

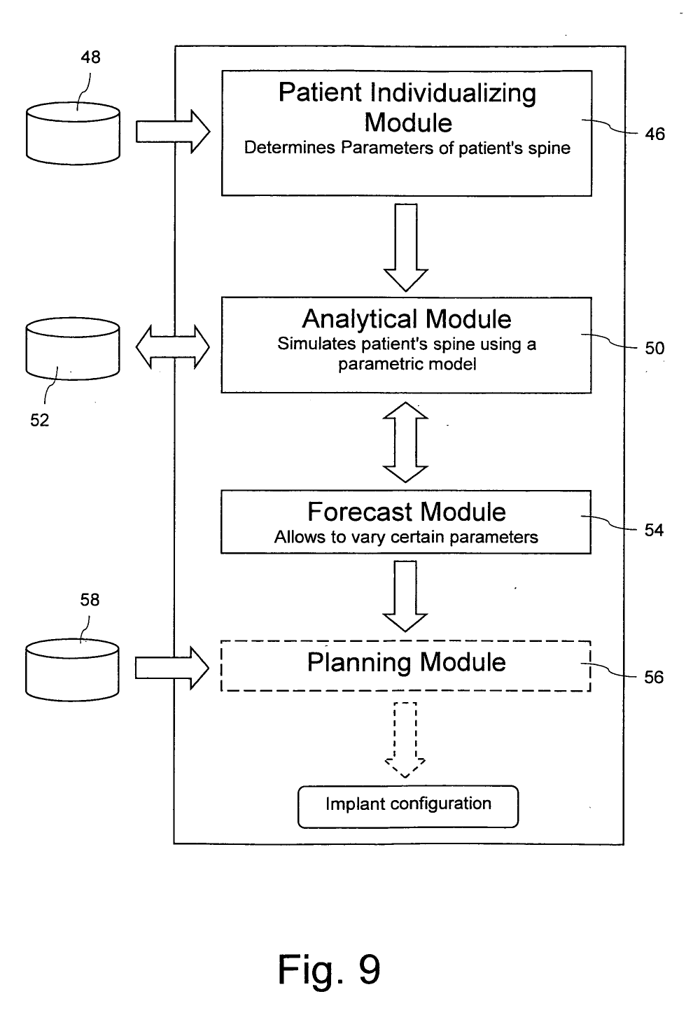

FIG. 8 schematically illustrates the basic function of the computer program. Biometric data of a patient 42 are fed to a computer 44. The computer 44 has also access to a data storage device 45 that stores at least a database containing biometric data of a large number of healthy persons or patients having at least one degenerated spine segment.

The computer program assists the surgeon in making a choice from the components that are required to assemble the prosthesis 20. This is illustrated at the bottom portion of FIG. 8, where two of the sets of components that are available are schematically illustrated as a line of boxes. Each box contains identical components of this set. In FIG. 8 the set denoted by SET26 comprises N boxes, with N being the total number of different upper supporting plates 26. The set denoted by SET30 comprises M boxes, with M being the total number of different joint elements 30. The assistance may basically consist of forecasting how the patient's spine will react if a particular implant was inserted at a particular location. The computer program may even be capable, if desired, to make a proposal for an optimum implant configuration. This is indicated by broken lines in FIG. 8 that are directed to specific boxes of the sets SET26 and SET30.

2.2 Main Program Modules

A patient individualizing module 46 determines parameters of the spine of the patient 42. To this end the patient individualizing module 46 has access to a patient's database 48 that contains images of the patient's spine in different spine positions and also patient related data such as age, sex, height and body mass index. The patient individualizing module 46 analyzes the images of the patient's spine and determines various parameters describing the patient's spine using a parametric spine model that will be described in more detail further below.

These parameters are supplied to an analytical module 50 which simulates the patient's spine using the parametric spine model. The analytical module 50 has access to a biometric database 52 which contains biometric data of a large number of other person. Preferably these biometric data are already stored in the form of parameters that can be used directly in the parametric spine model. The parameters of the other patients are used to refine or to supplement the parameters of the patient's spine that have been determined by the patient individualizing module 46.

A forecast module 54 allows the variation of certain parameters, in particular those parameters that are affected by inserting a particular implant. The forecast module 54 then simulates, by accessing the analytical module 50, what the effects of the parameter change are on the remaining parameters. Preferably these effects are graphically displayed so that the user can immediately understand how the patient's spine will react on any changes that may be brought about by changing one or more parameters.

The computer program may also include a planning module 56 which has access to an implant database 58 containing data of all available implants and components, for example data relating to the components that have been described above with reference to FIGS. 4 to 7. The planning module 56 simulates the effect that would be achieved by inserting different implants at a particular location of the patient's spine and comprises an assessment module which assesses whether the effect resulting from inserting the various implants are positive or negative. To this end the assessment module may use expert rules which quantify the positive or negative effects, similar to what is known from chess computer programs, for example. The result of these simulations is the output of a recommended implant configuration. The surgeon may then check, preferably by using a graphic display of the spine model, whether the recommended implant configuration complies with his experience and preferences.

2.3 Steps Performed by the Computer Program

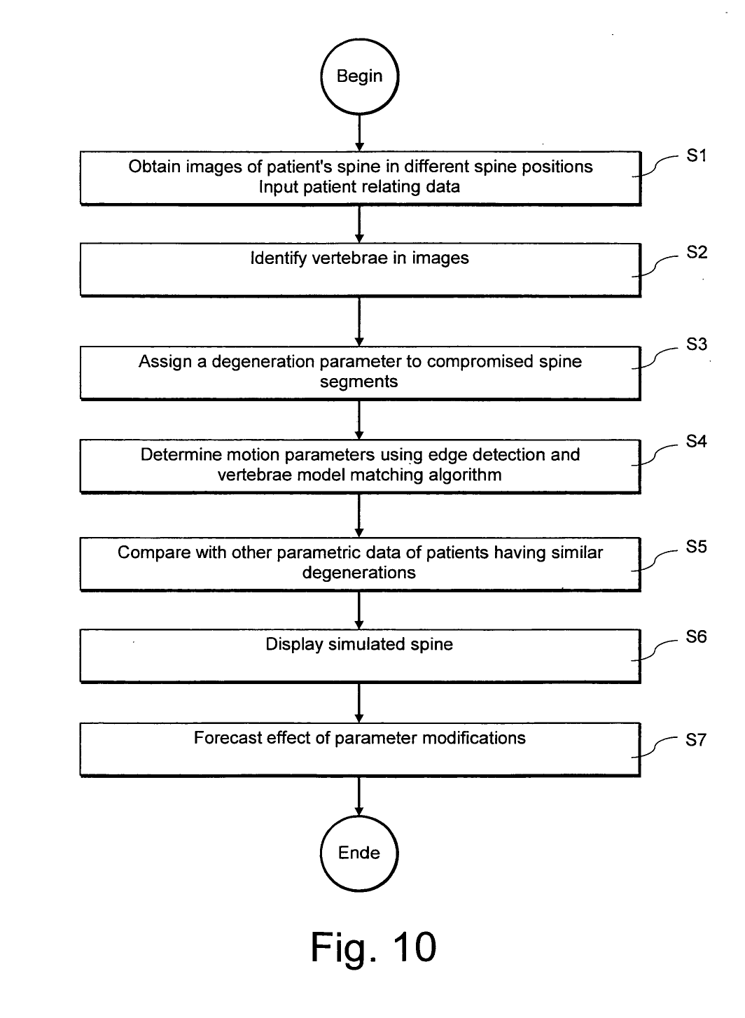

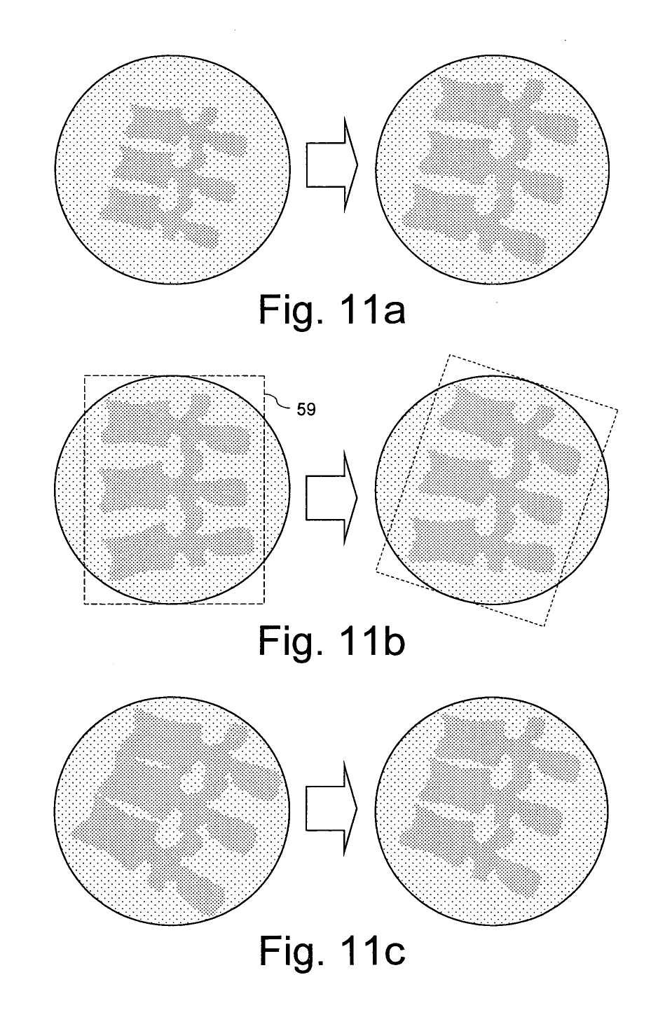

FIG. 10 is a flow chart of the main steps performed by the computer program. These steps will be explained in the following with reference to FIGS. 11 to 19. It is noted that these steps may be performed in a different sequence.

In a first step S1 images of the patient's spine are obtained in different spine positions. FIGS. 11a to 11c show, on the left hand side, sagittal digital X-ray images of three adjacent vertebrae of the patient's spine. In this illustration only three adjacent vertebrae are shown; as a matter of course, more than three or even all vertebrae of the patient's spine may be imaged. The images shown on the left of FIGS. 11a to 11c have been taken in different sagittal spine positions. In FIG. 11a it has been assumed that the spine is in a neutral position, i. e. the patient has been standing erect when taking the image. FIG. 11b shows the spine segment in a fully reclined position, i. e. the patient has extended his spine backward to a maximum extent. FIG. 11c shows the spine segment in a fully inclined position, which is obtained if the patient flexes his spine to a maximum extent by bowing his head towards his knees.

Often the images taken in different sagittal spine positions cannot be directly compared with each other because the patient has changed the distance from the X-ray detector. Then the same vertebrae shown in different images seem to have different sizes. This is illustrated on the left hand side of FIGS. 11a and 11b (here the size difference is exaggerated for the sake of clarity).

In order to be able to directly compare the images, it is then necessary to rescale the images such that same vertebrae have same sizes. Such a rescaling operation is illustrated in FIG. 11a. The vertebrae shown in the rescaled image on the right hand side have now the same size as the vertebrae in the image below in FIG. 11b. The scaling factor between different images may be deduced by determining the size of identical structures in different images.

Sometimes it may be desirable to obtain at least some images, for example a sagittal image and a coronal image of the spine in its neutral position, from which the orientation with respect to a reference direction, in particular the direction of gravity, can be determined. Sometimes the direction of gravity cannot be easily deduced from the images if blinds have been used by the X-ray assistant to avoid unnecessary exposure with X-rays. If these blinds are arranged obliquely, the software of the X-ray machine usually turns the image so that the edges of the image run either vertically and horizontally.

This is illustrated on the left hand side of FIG. 11b where a broken line 59 indicates the edges produces by the blinds in an image that has been turned by the X-ray machine so that these edges run either vertically and horizontally. However, the direction of gravity is not parallel to the vertical edges of the image. This will only be true after the image has been returned, as it is shown on the right hand side of FIG. 11b.

If it is not possible to easily determine from an image taken by the X-ray machine how it is oriented with regard to the direction of gravity of another reference direction, it may be necessary to use more sophisticated means as they are described further below in section 2.5 (Referencing).

Turning the images may also be useful if it is not necessitated by the use of blinds. Generally turning may help to reduce the image size, which is advantageous with a view to computer processing time.

As a matter of course, it may also be necessary to rescale and to re-turn the image, as it is shown in FIG. 11c for the fully inclined position.

After this rescaling and/or re-turning step the vertebrae shown in the images have identical sizes, and one of the images, for example the image showing the spine in the neutral position, may be shown in its correct orientation with respect to the direction of gravity or another reference direction.

It should also be noted that additionally a coronal image of the vertebrae may be taken (usually from a dorsal side) in a neutral position of the spine. Also for such an image a rescaling and/or re-turning operation may be performed so as to ensure that the different images of the vertebrae may be directly compared with each other.

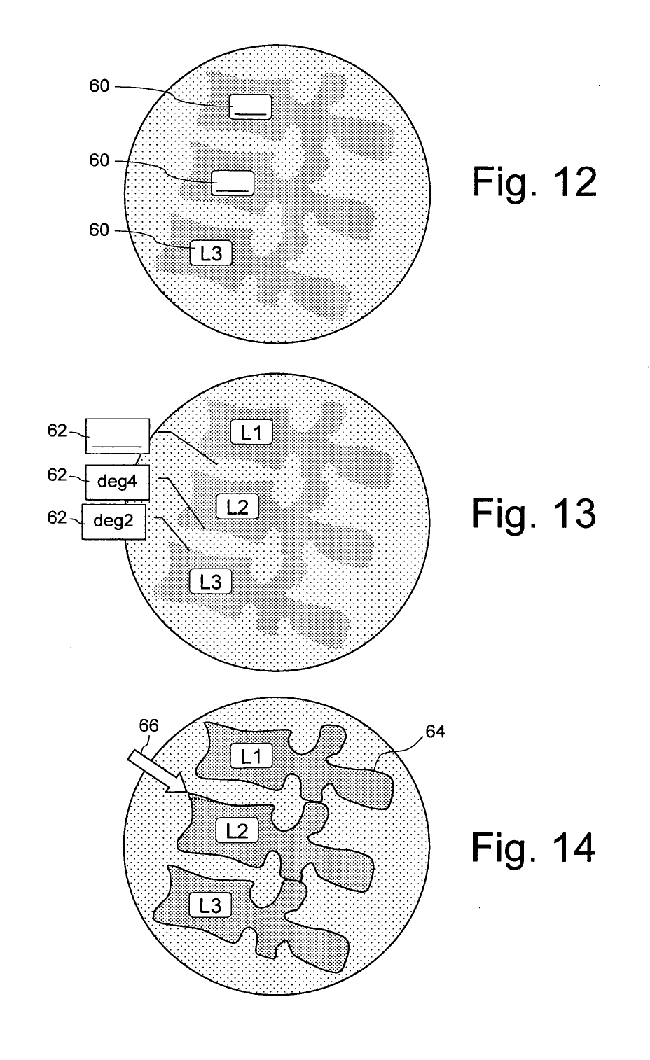

In a next step S2 the vertebrae shown in the images are identified. To this end the patient individualizing module 46 contains a routine that recognizes the vertebrae shown in the image using pattern recognition algorithms. This may be performed either in a fully automatic process or in a partially automatic computer assisted interactive process. Once the vertebrae in the image are recognized, prepared labels 60 are displayed in the image. At first all labels 60 are empty because the patient's individualizing module 46 cannot easily identify which vertebrae are shown in the image. However, the surgeon who has taken the image knows which vertebrae are shown. He then enters an identifier in the prepared label 60 of one of the vertebrae (see FIG. 12), for example the first sacral vertebra. The routine of the patient's individualizing module 46 then automatically assigns different identifiers to the other displayed vertebrae and displays the assigned identifiers in the image. This automatic completion is shown in FIG. 13 in which not only the vertebra L3, which has been labeled manually by the surgeon, but also the other two vertebrae L1 and L2 are correctly labeled. However, it should be noted that the user is free to move the labels at any time if they are not correctly assigned by the computer.

In an optional next step S3 degeneration parameters are assigned to compromised spine segments or their vertebrae. The patient individualizing module prompts to this end the user to assign degeneration parameters to those vertebrae or intervertebral disks that are degenerated. In FIG. 13 this is illustrated by input fields 62 in which the surgeon can input the degeneration parameters for those structures that he considers degenerated. In a next optional step S4 motion parameters using vertebrae detection and vertebrae model matching algorithms are determined.

To this end the patient individualizing module 46 first applies an edge detection and model driven energy minimization algorithm to the images so as to determine the outer shape of the imaged vertebrae. This is illustrated in FIG. 14 in which the detected edges 64 are shown by solid lines 64. The patient individualizing module 46 prompts the surgeon to confirm that the edges are correctly detected, but it may offer him also a possibility to modify the displayed edges 64. If the surgeon, by inspecting the displayed edges 64, believes that the edge detection algorithm has not properly detected the edge at a particular part of the image, for example because the image is blurred for whatever reason, he may manually correct the edge 64, as it is illustrated in FIG. 14 by an arrow 66. The arrow 66 is used to mark a certain portion of the edge 64 and to move the edge to its correct position. The program then recalculates and optimizes the internal spine model based on the manually set vertebra labels and edge contours (e.g. via measurement points).

The edges of the vertebrae may be depicted in different ways, for example as simple edge contours as shown in FIG. 14 or as corner points connected by straight lines. In one embodiment that edges are shown as colored fuzzy contours (clouds) to represent the degree of certainty of the edge detection. This assists the surgeon in identifying edges that require manual correction.

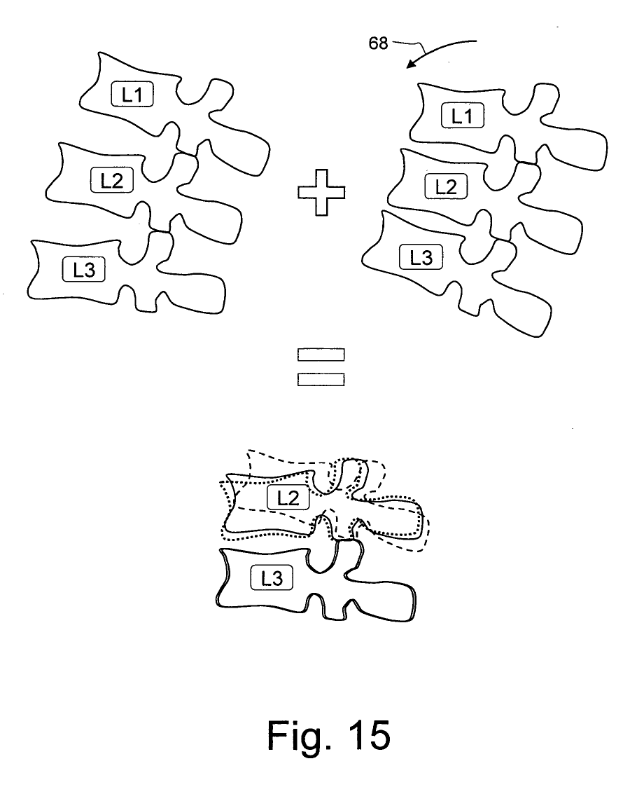

The patient individualizing module 46 further contains a routine which makes it possible, either manually or automatically, to match the vertebrae shown in the different spine positions so that it is possible to determine the ability to move relative to a neighboring vertebra. On the top of FIG. 15 the three vertebrae L1 to L3 are shown on the left hand side in their reclined position as shown in FIG. 11b, and on the right hand side in the inclined position as shown in FIG. 11c. If the right image is rotated as illustrated by an arrow 68, it is possible to match the lower vertebra L3, i. e. to move one vertebra over the other so that they coincide. This is shown in the lower portion of FIG. 15; for the sake of simplicity the upper vertebra L1 is not shown in this representation. The vertebra L2 shown with dotted lines 70 represents the inclined position, the broken line 22 the reclined position and the solid line 74 the neutral position as shown in FIG. 11a. The complex movement of the vertebrae as shown in FIGS. 11a to 11c is thus reduced to a representation in which one vertebra is fixed and a neighboring vertebra is moved to various positions that are indicated in FIG. 15 by lines 70, 72 and 74.

Preferably the process illustrated in FIG. 15 is repeated for all vertebrae of the spine, or at least for those vertebrae that are shown in the images.

The patient's individualizing module 46 further comprises a routine which determines a pivotal axis for pivotal movement between adjacent vertebrae and maximum pivotal angles for maximum reclination and maximum inclination. The position of the pivotal axis and the maximum pivotal angles are parameters that will be used by the analytical module 50 to simulate the patient's spine.

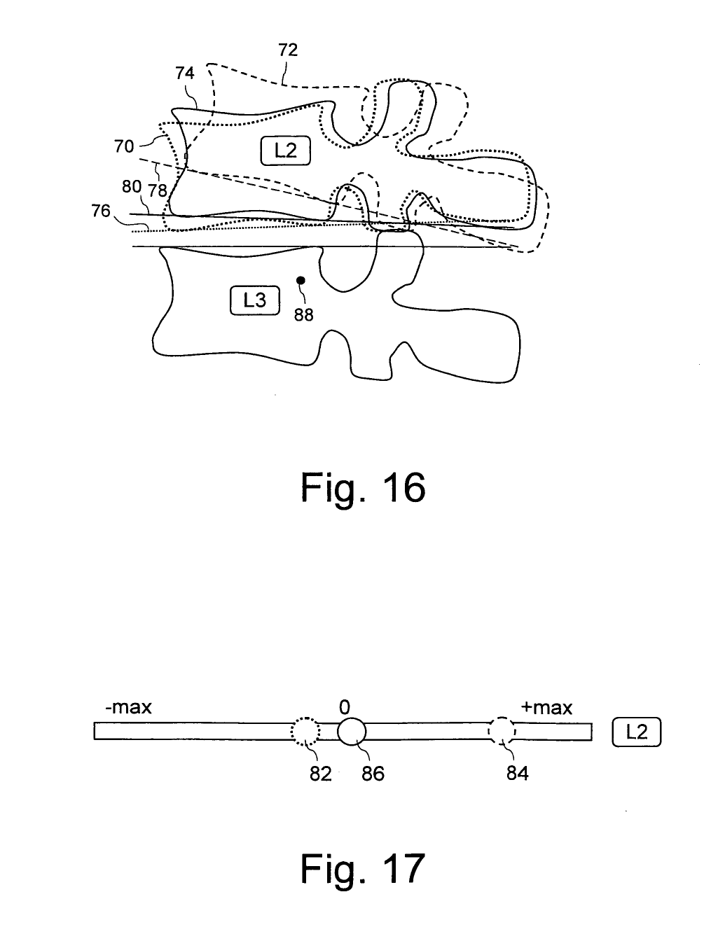

FIG. 16 is an enlarged view of the lower portion of FIG. 15. In addition to FIG. 15, a dotted line 76, a broken line 78 and a solid line 80 are shown that represent the pivoting angles in the inclined, reclined and neutral position, respectively. These lines are determined by the routine to compute the position of the pivotal axis and the maximum pivotal angles. In FIG. 16 it can be seen that the angle formed between the adjacent vertebrae L2, L3 in the neutral position (solid line 80) is slightly positive, in the inclined position (dotted line 76) slightly negative, and in the reclined position (broken line 78) significantly positive.

FIG. 17 illustrates this pivotal range of motion by three circles 82, 84, 86, wherein each circle represents a pivotal angle in the position of maximum inclination, the position of maximum reclination and the neutral position, respectively. All possible relative positions between the vertebrae L2, L3 can be represented by a circle which is arranged between the circles 82 and 84 representing the maximum angles. As a matter of course, this kind of illustration may be modified in many ways. For example, the range of motion may be represented by bars having different colors for inclination and reclination. These colors may become more intense until a maximum intensity is reached at the maximum angle of inclination and reclination, respectively.

In FIG. 16 the centre of motion is indicated by a black dot 88 which represents the pivotal axis for the pivotal movement of the vertebrae L2 and L3. It has to be noted that the relative movement of adjacent vertebrae often cannot be fully described as pivoting around a fixed pivotal axis. In the embodiment shown, for example, only the transition from the neutral position indicated by solid lines 74 to the inclined position indicated by the dotted line 70 can be accurately described by a pivotal movement around the centre of motion 88. However, from the neutral position to the reclined position indicated by broken line 72, the transition cannot be fully described by the pivotal movement around a fixed pivotal axis. Therefore the centre of motion 88 is often only important for small deviations from the neutral position. Outside this range of motion the centre of motion 88 cannot be regarded as fixed, but must be assumed as moving along a certain line which may be determined also by the patient individualizing module 46.

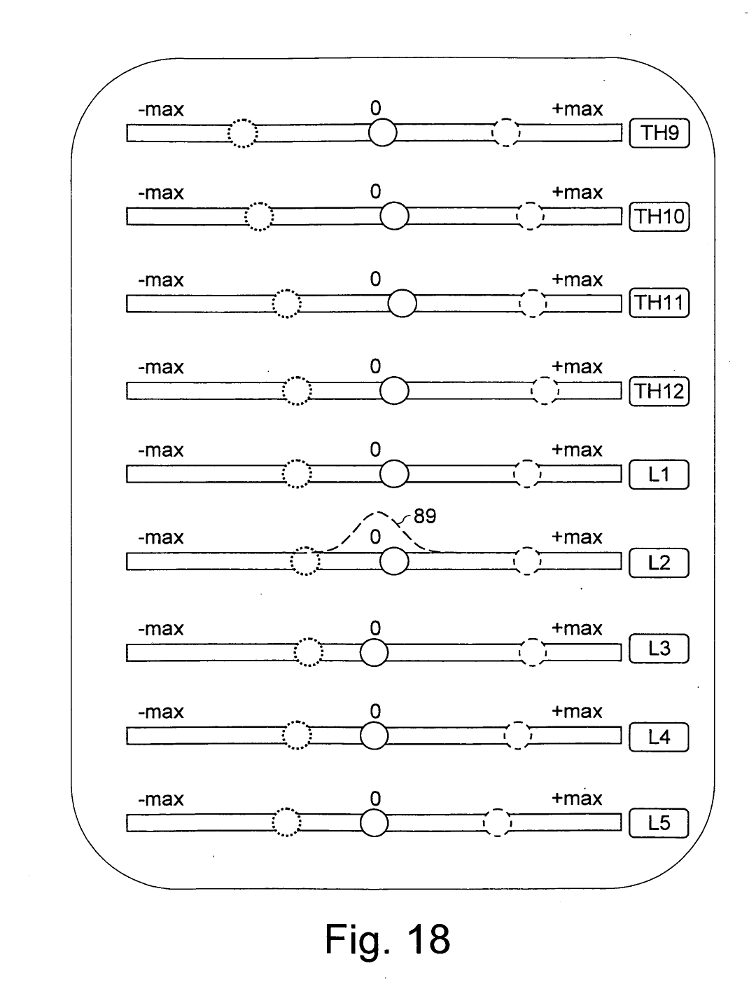

FIG. 18 is a graphic representation of a larger spine segment comprising nine adjacent vertebrae. For each vertebra the neutral position, the position if maximum inclination and the position of maximum reclination are graphically represented by circles indicated with solid lines, dotted lines and broken lines, respectively. From this representation it can be seen that the range of possible pivoting angles (i. e. sometimes also referred to as range of motion) usually differs from vertebra to vertebra, and also the angle of the neutral position is generally different for each vertebra.

In a step S6 the simulated spine is displayed. Preferably a graphic representation is used that makes it possible to identify easily the most relevant parameters at one glance. In the simplest case the graphic display looks like what is shown in FIG. 18, but there may also be additional information, for example the position of the centre of motions or the distances between adjacent vertebrae. Such additional information may also include statistical data. For example, it may be illustrated in the graph of FIG. 18 where the neutral position is located for other comparable persons having no degenerated spine segment. Such an illustration may include the display of a frequency distribution curve, as it is exemplarily shown for the vertebra L2 with a broken line 89.

In a next step S7 the effect of parameter modifications is forecast. If the interaction between adjacent vertebrae is known, it is possible to model the behavior of the spine segment. For example, in a position of maximum inclination the pivoting angles are given by the column of dotted circles shown in FIG. 18. If one is interested to know how the position of a vertebra changes if the adjacent vertebra is not moved to one of the three positions represented by circles in FIG. 18 for each vertebra, a simple interpolation may be carried out. This interpolation improves the more images of the patient's spine are available in different positions.

Another way of improving this simulation is to compare the data illustrated in FIG. 18 with corresponding data of other patients having similar degenerations. These data, which are stored in the biometric database 52, can be used to make a forecast also for modifications that go beyond a simple change of the pivotal angle within the range of motion. For example, if the distance between two adjacent vertebrae is changed in a certain fashion by inserting an implant, it is difficult to forecast the effect on the adjacent vertebrae solely on the basis of the images that have been taken from the patient's spine. In such a case comparative data may be helpful that relate to patients which had originally a spine with similar degenerations. If these comparative data comprise also images which have been taken from another patient after said distance has been changed in a similar fashion, these images can be used for improving the forecasts what the effects on the other vertebrae will be for the patient for whom the simulation is carried our. The comparison with other parametric data of patients having similar degenerations taking place in step S5 therefore contributes to the quality and reliability of the spine model which is used by the analytical module 50.

It is envisaged to add the biometric data obtained for the patient under consideration to the biometric database 52. Consequently, the size of the biometric database 52 increases with each additional patient. This results in a kind of self-learning effect, i.e. the more frequently the computer program is used, the better becomes the quality of the forecasts made by the computer program. The biometric data could be stored on a central server that obtains, for example via an online data exchange, these biometric data, from which all personal data relating to the patients have been deleted. The central server is then able to perform, on the basis of the anonymous biometric data, a parameter optimization by similarity search or by neuronal networks, for example. Such a centralized approach helps to build up quickly a database containing a huge amount of biometric data.

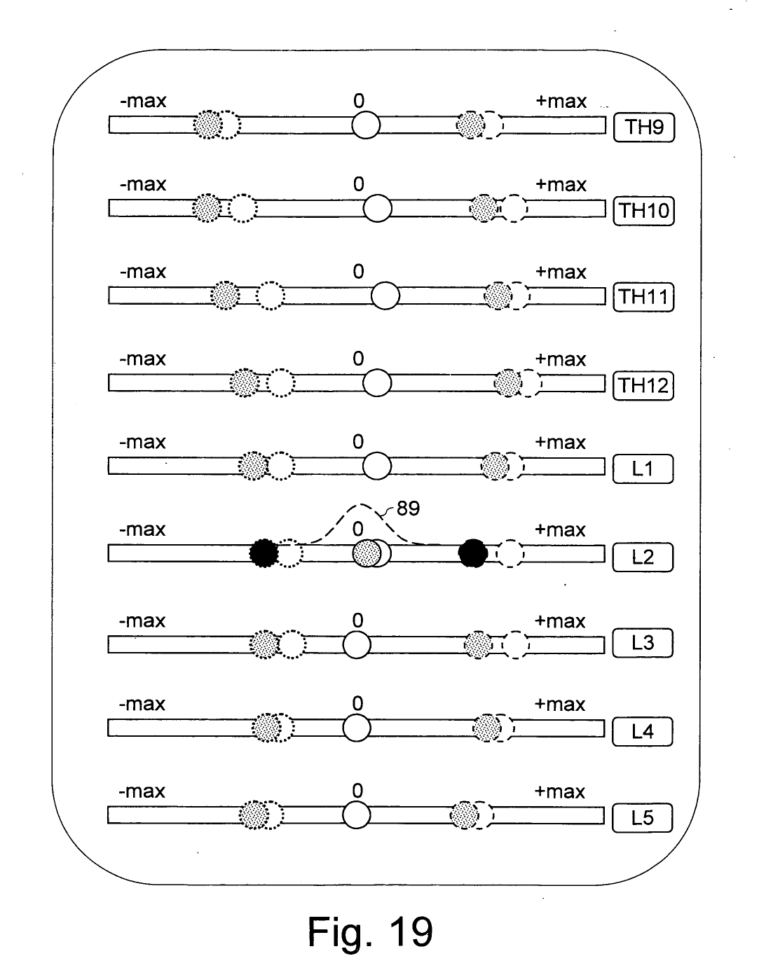

At the beginning of the forecast step S7, the surgeon varies one or more of the parameters that are displayed. FIG. 19 shows the graphic display of FIG. 18, but with two additional black circles which represent modifications that have been input by the surgeon, perhaps with a view to the statistical data displayed with curve 89. The black circles represent a modified range of motion which may be accomplished by a particular prosthesis whose pivotal range of motion is limited by a particular choice of components, as it has been explained above with reference to FIGS. 3 to 7. The forecast module 54 now forecasts, using the spine model applied by the analytical module 50, how this modified range of motion will affect the range of motions of the adjacent vertebrae. In the illustration of FIG. 19 it is assumed that the reduction of the maximum reclination (black circle on the right side) also reduces the maximum reclination angles of the adjacent vertebrae. This may be helpful for avoiding too large reclination angles, as they may have occurred at the vertebra below, because a too large reclination angle may have adverse effects on the intervertebral disk and may foster further degenerations of the vertebra.

In a similar manner the surgeon may try out other configurations of the implant and check whether the effects on the other vertebrae will have an overall positive or negative impact on the spine segment under consideration.

2.4 Extension to 3D Simulation

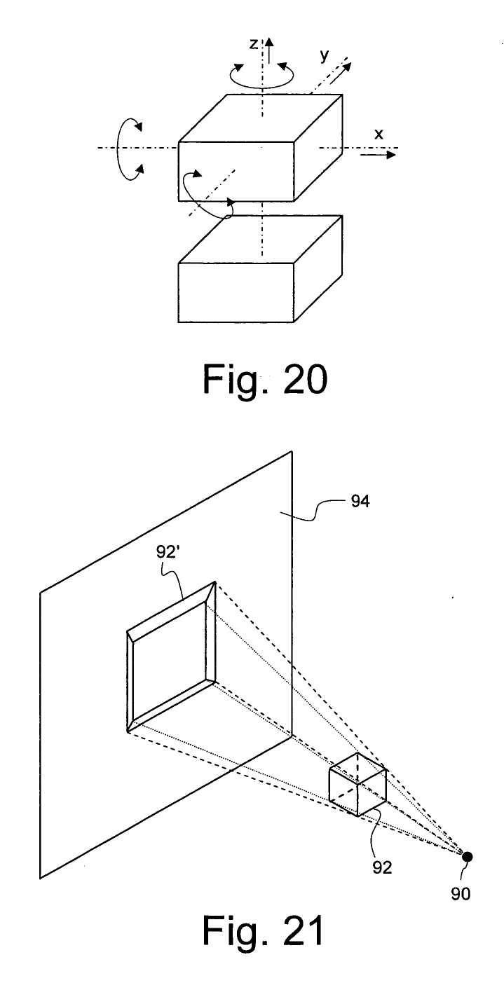

In the foregoing it has been assumed that the computer program simulates only the motion of the vertebrae in a sagittal plane. For many applications, however, it is advantageous to consider also the other degrees of rotational and translational freedom the vertebrae have. FIG. 20 illustrates how two adjacent vertebrae represented by parallelepipeds may perform more complex movements by rotations and translations along three orthogonal axes X, Y, Z.

For fully understanding the complex motion of the vertebrae, not only images in a sagittal plane, but also in an orthogonal coronal plane should to be taken. Often six images will suffice, namely three images (neutral, inclined, reclined) for a sagittal plane and three images (neutral, laterally flexed to the right and to the left) in a coronal plane.

In the simplest case the images of the patient's spine in different spine positions are 3D images, which may have be obtained using CT or MRT imaging techniques. Then the extension to a 3D modeling is comparatively straightforward.

Usually, however, only 2D images of the patient's spine in different spine positions are available. In such cases an extension to 3D modelling requires that the position of the vertebrae can be determined solely on the basis of the 2D images shown in FIGS. 11a to 11c. However, since a 2D image is usually a central projection of the vertebrae on a screen, the orientation of the vertebrae in three dimensions can be computed if the locations of the X-ray source and the X-ray detector and also the shape of the vertebrae are known. FIG. 21 shows the situation that X-rays emerge from a point X-ray source 90 and project a vertebra, which is represented by a parallelepiped 92 having a known shape, on an X-ray detector 94. From the image 92′ of the vertebra it is possible to determine the orientation of the vertebrae 92 in three dimensions.

The shape of the vertebrae may be determined for a specific patient using a single 3D image of his spine. Alternatively, comparative data stored in a database may be used to this end. In this context it should be noted that degenerations usually affect only certain parts of the vertebrae. With regard to the remaining parts, the shapes of the vertebrae are almost the same for patients being similar in terms of sex, age, height and body mass index. Therefore it is usually sufficient to assume standardized shapes of the vertebrae and to perform the determination of the vertebra's orientation only on the basis of those parts which are usually not subject to degeneration.

If for three different spine positions, for example neutral, inclined and reclined, images are taken both in a sagittal and a coronal plane, both sets of images allow to determine the orientation of the vertebrae. By comparing the orientations determined from the sagittal and the coronal images, the accuracy can be improved, because two statistically independent measurements have been carried out.

Once the 3D orientations of the vertebrae are determined for the three different configurations shown in FIGS. 11a to 11c, the modelling of the spine described above for two dimensions can be simply extended to the other two dimensions, too. In such an extended model the set of parameters that describe the position and movements of the vertebrae is increased correspondingly. In a first approximation the pivoting movements in the different planes can be regarded as being independent from one another so that a pivoting movement of one vertebra does not cause a pivoting movement of an adjacent vertebra around an orthogonal pivotal axis. In a more refined model also interactions between movements in different planes may be considered.

2.5 Referencing

If the patient carries a plumbline while the images are taken, there is also an absolute reference in the images which makes it possible to determine the exact orientation of the vertebrae in 3D space. A similar effect may be achieved if the patient carries a belt containing a level. All parameters such as pivotal angles or the positions of the centers of motions may then be given relative to the reference direction determined by the plumbline or the level. Other and more sophisticated approaches to obtain a reference direction in images are described in German patent application DE 10 2010 026 934.4.

In one embodiment the computer program uses for each vertebra a coordinate system. The origin of this coordinate system should then be associated to a particular point of the vertebra that is usually not be affected by degenerations. Here the computer program selects as origin of the coordinate system a point which is located within the pedicle domes. This point may be defined, for example, by the intersection of an axis 96 of circular symmetry, which runs through the pedicle domes 98 shown in FIG. 2, and a coronal plane of symmetry which runs through the center between the pedicle eyes and the processus spinosus.

3. Fusion Implant

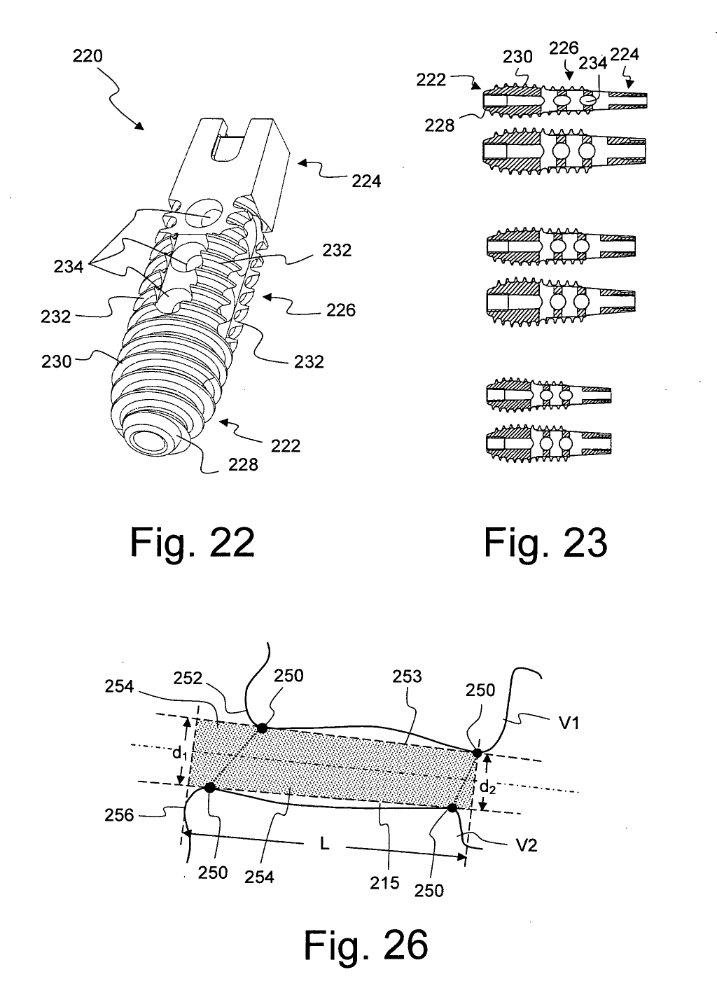

FIG. 22 is a perspective view of a fusion implant 220 as it is described in US 2007/055374 A1 filed by one of the present inventors. The full disclosure of this earlier application is incorporated herein by reference.

The fusion implant 220 has a head portion 222, a base portion 224 and a central portion 226 extending between the base portion 224 and the head portion 222.

The head portion 222 tapers down towards a rounded tip 228 and is provided with an external thread 230 that reaches down over the central portion 226 to the base portion 224.

The central portion 226 is formed by four struts 232 that are separated from each other by openings 234.

The base portion 224 has a square or rectangular cross section and also tapers down towards the end of the implant 220, as it can best be seen in the cross sections through differently sized implants 220 shown in FIG. 23.

As it has been mentioned above, in a healthy human spine the joints formed by pairs of adjacent vertebrae have properties that significantly differ along the spinal column. Therefore the fusion implant 220 has to be adapted to these different properties. The surgeon should be able to select, for a particular compromised intervertebral disc compartment, from a set of differently sized implants a fusion implant 220 that fits best into the intervertebral disc compartment and also restores the original configuration of the compromised spine segment as well as possible.

FIG. 23 shows six differently sized fusion implants 220 that may belong to a set of implants from which the surgeon can select. As can be seen in FIG. 23, the implants 220 of this set have different lengths. Implants 220 of equal length have different diameters along their longitudinal axis, which, in turn, results in different angles formed between the head portion 222 and the base portion 224. This angle is crucial because it defines the angle between the vertebrae to be fused.

It is difficult for a surgeon to make an optimum selection from such a set of different fusion implants 220 merely on the basis of experience and some biometric data relating to the specific patient. In the following an embodiment of a computer program will be described that supports the surgeon in his task of selecting a suitable fusion implant 220 so that the patient will be relieved from pain also in the long term. This implies that other portions of the spinal column are not unduly strained, which would ultimately result in new discomforts and pains. It is to be understood that in principle the same steps may be taken to determine the configuration of a non-fusion implant.

3.1 Computer Program

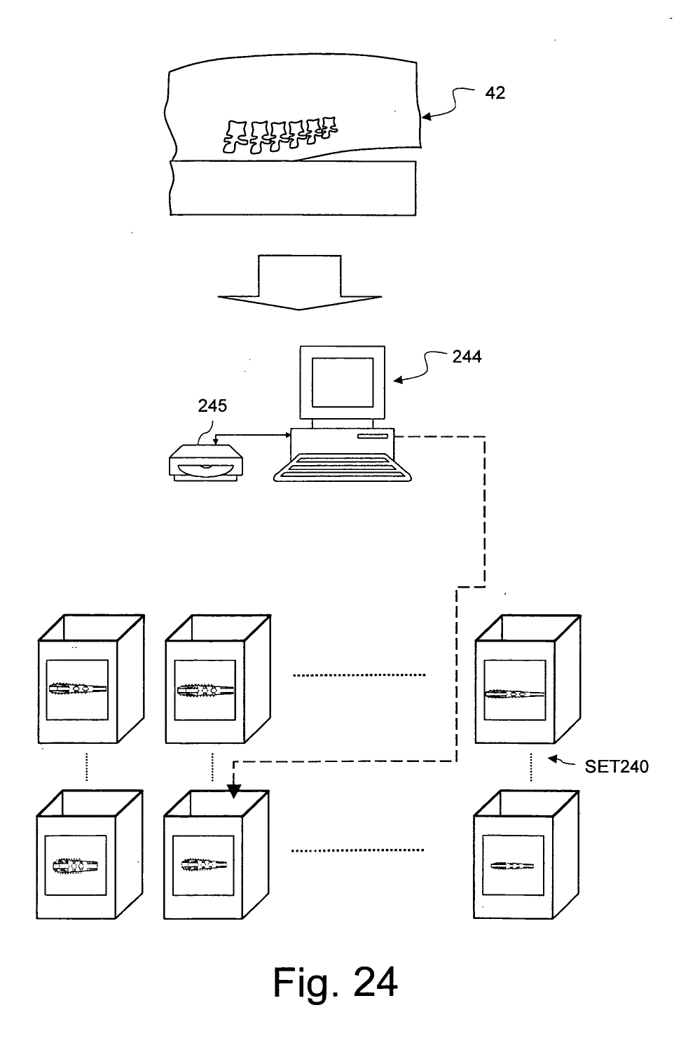

FIG. 24 schematically illustrates, in a representation similar to FIG. 8, the basic function of the computer program. Biometric data of a patient 42 are fed to a computer 244. The computer 244 has also access to a data storage device 245 that stores at least biometric data, or quantities (such as mean values, for example) derived from such data, of a large number of persons having no degenerated spine segments.

The computer program assists the surgeon in making a selection of a suitable fusion implant 220 from a set of fusion implants having different sizes. This is illustrated at the bottom portion of FIG. 24, where a set SET240 of fusion implants having different geometrical properties is schematically illustrated as a number of boxes. Each box contains identical fusion implants 220, but different boxes contain different fusion implants 220. The assistance provided by the computer program may basically be a forecast how the patient's spine will react if a particular fusion implant 220 out of the set SET240 is inserted at a particular location along the spinal column. The computer program may even be capable, if desired, to make a proposal for an optimum fusion implant 220. This is indicated by a broken line in FIG. 24 that is directed to one specific box of the set SET240.

3.2 Steps Performed by Computer Program

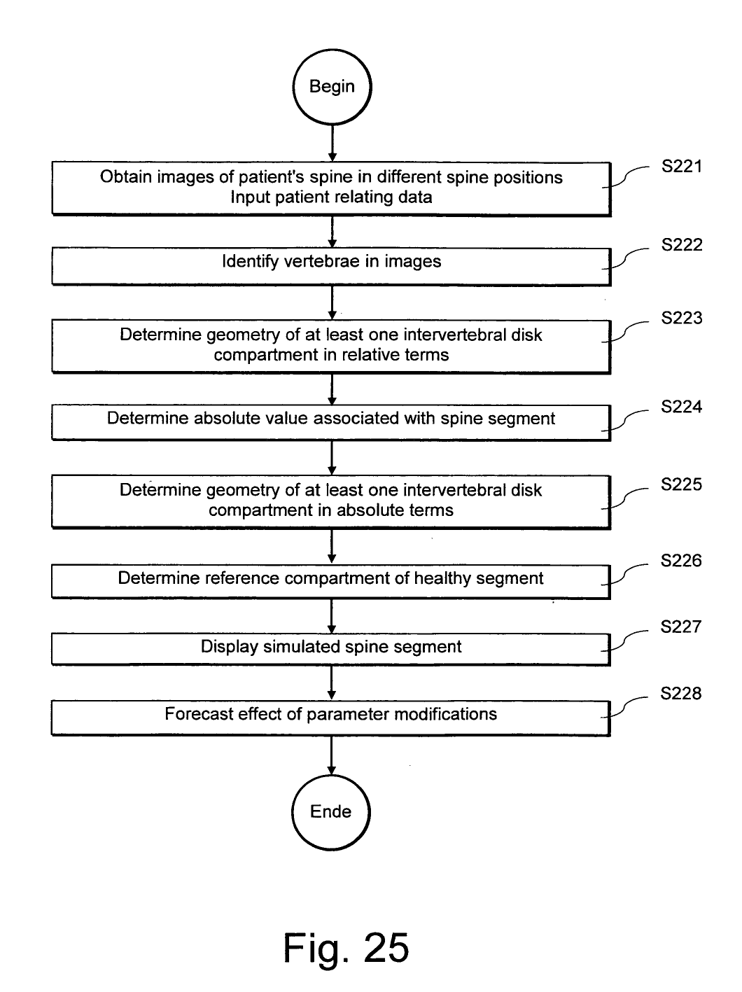

FIG. 25 is a flow chart of the main steps performed by the computer program. These steps will be explained in the following with reference to FIGS. 26 to 28. It is again noted that these steps may be performed in a different sequence.

The first two steps 5221 and 5222 in this embodiment are identical to the steps S1 and S2 as shown in FIG. 10 for the first embodiment. Reference is therefore made to FIGS. 11 and 12 and to the description of these figures.

In a third step S223 the geometry of at least one intervertebral disc compartment is determined in relative terms. How this is accomplished will be explained in the following with reference to FIG. 26 which shows an intervertebral disc compartment that is formed between two adjacent vertebrae V1, V2.

On a computer screen the computer displays four points 250 that are located on edges 252 of the vertebrae V1, V2 that have been detected by an edge detection algorithm. The computer positions the four points 250 such that they are in immediate vicinity to the intervertebral disc compartment 215. Furthermore, the four points 250 are arranged at the lateral sides of an isosceles trapezoid 254 that defines in a sagittal plane the geometry of the intervertebral disc compartment 215. The points 250 should be located such that a fusion implant 220 engages the adjacent vertebrae V1, V2 at the points 250. Thus the points 250 do not denote the corners of the vertebrae V1, V2 (i. e. the locations where the edges have their smallest radius of curvature), but positions where a trapezoid 254 would touch the vertebrae V1, V2.

If the algorithm provided by the computer fails to correctly position the points 250 automatically, the surgeon may move one or more of the points 250 to positions that he deems correct. The geometry of the trapezoid 254 will then be automatically adjusted if the surgeon moves one of the points 250 along a direction which does not coincide with lateral sides 253 of the trapezoid 254.

For the compromised intervertebral disc compartment it is necessary to determine also the length L of the trapezoid 254, because this defines the length an implant 220 to be inserted should have. To this end the algorithm used by the computer determines the length L of the trapezoid 254 by using additional geometrical information obtained from the detected edges. For example, the front end of the trapezoid 254 having a diameter d₁ is determined such that it is aligned with a front edge 256 of the lower vertebra V2. The rear end of the trapezoid 254 having a diameter d₂ is determined such that it coincides with the rear point 250 on the upper vertebra V1. Again, the surgeon may be prompted by the computer program to modify the suggestions made by the algorithm.

From the length L and the four points 250 the diameters d₁ and d₂ and also the angle formed between the lateral sides 253 can be easily determined.

In this manner the dimensions of the compromised and at least of the immediately adjacent intervertebral disc compartments 215 are measured. Preferably at least 5 to 7 adjacent intervertebral disc compartments are measured in the same manner.

In a next step S224 an absolute value associated with the spine segment is determined. Such a determination is necessary because usually the absolute dimensions of the vertebrae V1, V2 cannot be immediately obtained from two dimensional sagittal X-ray images. Thus the determinations explained above with reference to FIG. 26 in step S223 can usually be performed only in relative terms.

One possibility to obtain an absolute value associated with the spine segment is the use of 2D or 3D CT images. Another approach would be to apply a meter rule or a similar scale to the patient when the X-ray image is taken. The meter rule is then also projected on the X-ray detector, and its image and the X-ray images enable the surgeon to perform a calibration of the measurement results that have been obtained before in step S223.

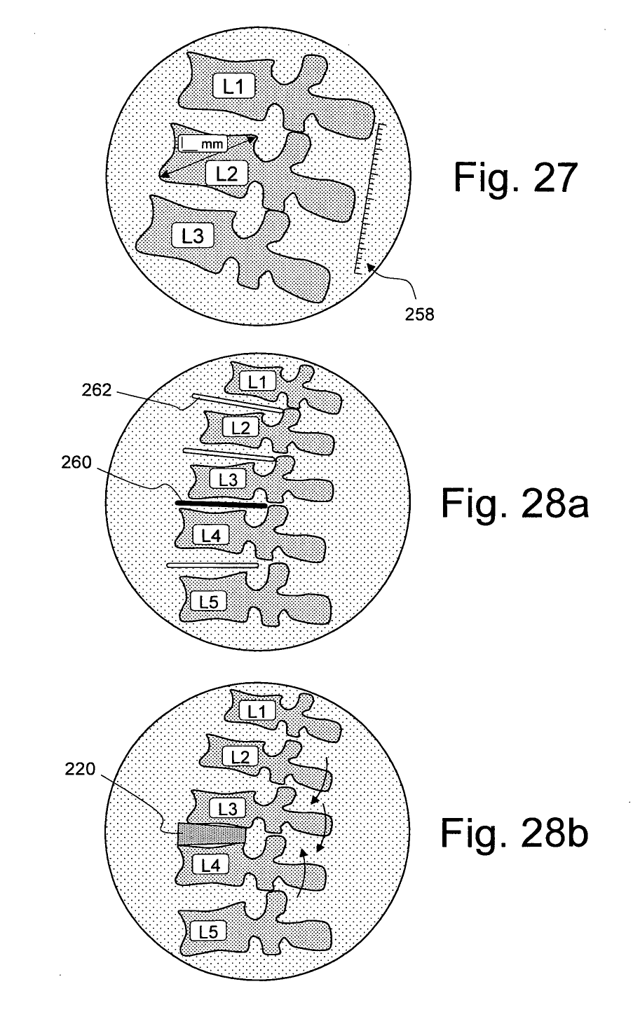

In this context the surgeon may simply measure on the X-ray image a certain dimension, for example the diagonal of a certain vertebra L2, as it is shown in FIG. 27. By comparing this dimension with the image 258 of the meter rule, the surgeon is able to enter the absolute value of the selected dimension, here the diagonal of the vertebra L2. Then the computer program is able to determine the geometries of the trapezoids 254 representing the intervertebral disc compartments also in absolute terms.

A third approach may be to use detailed information on the acquisition geometry when the X-ray image was taken. Such information may include the position of the patient (for example footsteps placement) relative to the X-ray source and the X-ray detector.

In a next step S226 the compromised intervertebral disc compartment, where the fusion implants 220 shall be implanted, and reference compartments which are not in a compromised state are determined by the surgeon. In FIG. 28a it is assumed that the intervertebral disc compartment between the vertebrae L3 and L4 is compromised, i. e. one of the fusion implants 220 shall be implanted there. This intervertebral disc compartment is therefore indicated by a colored bar 260. Of course, a similar way of identification, for example a marker at the boarder of the window right or left to the image, may be used instead. Non-compromised compartments in the neighborhood of the compromised compartment may also be highlighted, for example by using bars 262 in a different color.

In a next step S227 the simulated spine segment is displayed. The surgeon is now able to displace one of the vertebrae L3, L4 that define the compromised intervertebral disc compartment indicated by the bar 260.

For the compromised compartment indicated with the bar 260 the computer program proposed a suitable fusion implant 220 It can be seen in this illustration that this proposed fusion implant 220 has been graphically inserted between the two vertebrae L3, L4 and thus replaces the bar 260 indicating the compromised compartment. As a matter of course, the surgeon may be able to discard this proposal immediately and replace the proposed implant 220 by another implant that he deems more suitable.

When proposing a suitable implant 220, the computer program may perform the following steps:

First statistical data of spines of other persons are fetched from the biometric database 52. In one embodiment these data comprise the diameters d₁ and d₂ and the length L of the intervertebral disc compartments as shown in FIG. 26. Other sets of data may be used instead as long as they suffice to determine the geometry (i.e. shape and size) of the intervertebral disc compartments in the sagittal plane. Preferably these data are obtained from healthy persons. However, also data from patients having a compromised spine segment may be used, because further away from the compromised spine segment its impact on the other segments is so small that it can be neglected. In other words, in this approximation these patients may be considered as healthy patients.

In a next step the corresponding data describing the shape and size of the patient's non-compromised segments are compared with the data fetched from the database 52. Those data sets, for which a sufficient similarity to the patient's data is found, are identified. For these data sets the data relating to the specific intervertebral disc compartment, for which the patient requires fusion, are used to compute a mean shape and size of the compartment. Now it is assumed that the patient's compromised intervertebral disc compartment had, before its degeneration began, approximately the same shape and size as comparable compartments of healthy persons. Consequently it is determined that the patient's compromised compartment should have the same shape and size as it has been determined for the comparable healthy persons.

Generally the geometry of the intervertebral disk compartment determined in this manner will be different from the geometry before the surgery commences. This implies that the adjacent vertebrae L3, L4 have to be rearranged or, in other word, the angle change caused by the surgery has to be distributed among the adjacent segments. The computer program computes now the angle difference between the original state and the state with the inserted implant 220 and then makes a prediction how the neighboring vertebrae will react on this change. It is to be noted that this process relates to a particular posture (usually the neutral posture), and it has to be distinguished from a prediction how the spine will react if it is bend.

In this embodiment the computer distributes the angle change among the neighboring vertebrae in proportion to the range of motion that has been determined before for these vertebrae. The range of motion is defined as the difference between the pivotal angles for maximum extension and maximum flexion, as it has been explained above with reference to FIG. 17. In other words, those vertebrae which have a large pivotal range are considered by the algorithm to be capable to compensate more of the angle change, which has been caused by the anticipated insertion of the fusion implant 220, than vertebrae having a smaller ability to pivot.

The distribution of the angle change among healthy intervertebral disc compartments is usually performed not for all, but only for a number of vertebrae in the neighborhood of the affected spine segment. This is based on the conception that not all vertebrae will rearrange after the anticipated insertion of the implant 220. Some vertebrae do not pivot at all or only very little. For example, the vertebrae of the thoracic spinal column 18, to which the ribs are attached, may be regarded as fixed. Also the sacrum S of adults, which is fixedly connected to the pelvic, may be regarded as fixed in this context, although small pivotal movements of the sacrum S to compensate for angle changes of the lumbar vertebrae have been observed. If the fusion implant is to be inserted within the lumbar region of the spinal column, the vertebrae Th12 (lowest vertebra of thoracic spinal column 18) and the sacrum S may thus be regarded as fixed. Only the vertebrae between these two elements whose pivotal position is assumed to be fixed are allowed to compensate for any angle change which has been caused by the insertion of the fusion implant 220. Similar considerations apply to the cervical spinal column 16.

In FIG. 28b it is assumed, for the sake of simplicity, that the two vertebrae L1, L5 are kept in the same pivotal position, and only the vertebrae L2, L3 and L4 are able to perform movements (indicated by arrows) that compensate the angle change introduced by the anticipated insertion of the implant 220.

If the surgeon comes to the conclusion that the adjusting movements of the vertebrae L2 to L4 are likely to produce undue strains on these vertebrae, he may decide to simulate the situation for a different fusion implant 220. An indication for such undue strains is often if the vertebrae L2 to L4 will move to positions that are very close to the end positions which they have in a fully extended or a fully flexed state of the spine.

The surgeon can monitor this by using a graphic representation as is it shown in FIGS. 18 and 19 for the case of non-fusion implants. The only difference to this representation is that the range of motion will be zero for the operated segment, i.e. there would be only one black dot at the position which represents the angle fixed by the implant. In such a representation an overstrain of adjacent vertebral joints can be recognized if in one of the lines associated with different vertebrae the middle circle indicating the neutral position moves very close to the position of maximum inclination or flexion.

3.3 Result Screen

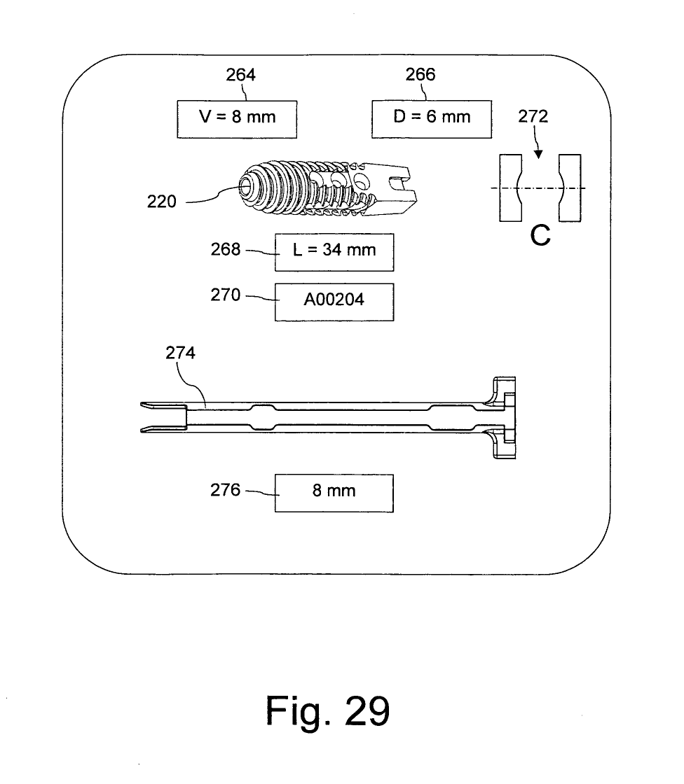

FIG. 29 shows how the results may be output by the computer program on a screen or a printout. The recommended implant 220 is shown with its real dimensions. These dimensions, for example the ventral and dorsal diameters d₁, d₂ and the length L (see FIG. 26), are additionally indicated in millimeters at 264, 266 and 268, respectively. An article number that uniquely identifies each implant from the set SET240 of implants may be indicated as well at 270.

Here it is assumed that the base portion 224 of the implant 220 is rectangular. This results in two different dorsal diameters d₂ that can be obtained with a single implant 220 depending on its orientation in the intervertebral disc compartment. The illustration at 272 informs the surgeon in which orientation the implant 220 has to be inserted into the intervertebral disc compartment. In this context reference is made to DE 20 2010 011 773 U1, which describes in more detail various aspects that are associated with the use of implants 220 having a rectangular base portion 224. The full disclosure of DE 20 2010 011 773 U1 is incorporated herein by reference.

In a lower portion of the screen a sleeve 274 is shown in a true-to-scale representation with the diameter of the sleeve 274 indicated at 276. The sleeve 274 is recommended by the computer program for inserting the implant 220 shown above into the intervertebral disc compartment. If a system of differently sized sleeves and rods having elliptical head portions is used to distract the intervertebral disc compartment stepwise, as it is described in the aforementioned DE 20 2010 011 773 U1, the computer program may also show the sequence of sleeves and rods that the surgeon may use in this process.

### Other Modifications

In one embodiment also the distances between the pedicles of the adjacent vertebrae are considered. This may be particularly important in the case of fusion implants.

If certain biometric data, for example forces or distances between adjacent vertebrae 20, can only be measured during the implant surgery, the computer program may be able to process such data and take them into account when modeling the patient's spine. To this end measuring instruments may be used that are capable of feeding data directly to the computer, as they are disclosed in WO 2010/037558 A2. With this instrument it is possible to obtain a functional dependency of the forces that are exerted by the vertebrae, which are to be connected by a fusion or non-fusion implant, depending on the distance between these two vertebrae. Since the forces prevailing between the two vertebrae should be within a certain range, it is possible to determine from such a function a range for the distances that should be established between the two vertebrae with the help of the implant.

In section 3.2 it has been mentioned that to some extent also the sacrum S may pivot as a result of an angle change that is produced by the introduction of a fusion implant. If this shall be considered, too, it is necessary to include the sacrum S in the vertebrae among which the angle change produced by the insertion of the fusion implant has to be distributed. Then not the sacrum S, but the plane of the femoral joints will be considered as being fixed.

The above description of the preferred embodiments has been given by way of example. From the disclosure given, those skilled in the art will not only understand the present invention and its attendant advantages, but will also find apparent various changes and modifications to the structures and methods disclosed. The applicant seeks, therefore, to cover all such changes and modifications as fall within the spirit and scope of the invention, as defined by the appended claims, and equivalents thereof.

1-85. (canceled)

86. A computer program stored or running on a medium and configured to cause a computer to perform the following steps:
a) accessing biometric data which relate to a spine of a patient, the spine having at least one compromised spine segment;
b) accessing a database in which geometries of intervertebral disc compartments of other persons are stored;
c) identifying other persons having intervertebral disc compartments which have a similar geometry as corresponding intervertebral disc compartments of the patient in non-compromised spine segments, wherein the similarity is determined by an algorithm;
d) from the persons identified in step c), using a geometry of the intervertebral disc compartment, which corresponds to the intervertebral disc compartment of the compromised segment of the patient, to compute a mean geometry;
e) determining a configuration of an implant that will, if inserted into the intervertebral disc compartment of the compromised spine segment of the patient, change its geometry such that it is at least substantially identical to the mean geometry determined in step d).

87. The computer program of claim 86, wherein the biometric data comprise image data obtained from images of the patient's spine in different spine positions, the images having been taken by using medical imaging techniques.

88. The computer program of claim 87, wherein the different spine positions comprise a neutral position, a fully extended position and a fully flexed position.

89. The computer program of claim 87, wherein the computer program is configured to cause a computer to graphically display at least one of the images or a portion thereof, and to cause the computer to perform the following steps:
i) prompting a user to assign a first identifier to one of a plurality of vertebrae shown in the at least one image;
ii) automatically recognizing the vertebrae shown in the at least one image using a pattern recognition algorithm;
iii) automatically assigning different identifiers to other vertebrae displayed in the at least one image;
iv) displaying the identifiers assigned in step iii) in the at least one image.

90. The computer program of claim 89, wherein the computer program is configured to cause the computer to prompt the user to assign a degeneration parameter to at least one vertebra and/or to at least one intervertebral disk.

91. The computer program of claim 87, wherein the computer program is configured to cause a computer to determine edges of vertebrae shown in at least one of the images using an edge detection algorithm.

92. The computer program of claim 91, wherein the computer program is configured to cause the computer to display the edges and to offer the user to modify the displayed edges.

93. The computer program of claim 87, wherein the computer program is configured to cause a computer to offer a user to determine a geometry of an intervertebral disk compartment, which is located within the compromised spine segment, by arranging four points on or in close vicinity to edges of vertebrae which are shown in at least one of the images and between which the intervertebral disk compartment is formed.

94. The computer program of claim 93, wherein the computer program is configured to cause the computer to display the four points in the at least one image in such a manner that the four points are always located on a variable isosceles trapezoid.

95. The computer program of claim 93, wherein the computer program is configured to cause the computer to assign a measured reference value to a geometric parameter of a vertebrae that is displayed in the at least one image.

96. The computer program of claims 94, wherein the computer program is configured to cause the computer to assign a measured reference value to a geometric parameter of a vertebrae that is displayed in the at least one image, and wherein the computer program is configured, if running on the computer, to cause the computer to compute the real dimensions of the isosceles trapezoid based on the measured reference value.

97. The computer program of claim 87, wherein the computer program is configured to cause a computer to determine, for at least some of the vertebrae shown in the images, the ability to move relative to a neighboring vertebra, and to determine parameters describing this ability.

98. The computer program of claim 97, wherein the computer program is configured to cause the computer to determine pivotal angles for maximum extension and maximum flexion.

99. The computer program of claim 97, wherein the computer program is configured to cause the computer to determine a pivotal axis for pivotal movements between adjacent vertebrae.

100. The computer program of claim 98, wherein the computer program is configured to cause the computer to determine a range of motion for at least some of the vertebrae shown in the images, wherein the range of motion is defined as a difference between pivotal angles for maximum extension and maximum flexion of the spine.

101. The computer program of claim 100, wherein a user is enabled to produce an angle change between two adjacent vertebrae, and wherein the computer program causes the computer to distribute the angle change among a plurality of adjacent vertebrae.

102. The computer program of claim 101, wherein the computer program causes the computer to distribute the angle change among the plurality of adjacent vertebrae in proportion to the range of motion determined for these vertebrae.

103. The computer program of claim 97, wherein the computer program is configured to cause the computer to compare the parameters relating to the patient to corresponding parameters that relate to other persons and are stored in a database.

104. The computer program of claim 103, wherein the computer program is configured to select the other persons by comparing degeneration parameters of the other persons to degeneration parameters of the patient.

105. The computer program of claim 97, wherein the computer program is configured to cause the computer to modify the parameters of the patient by statistically analyzing parameters of other persons.

106. The computer program of claim 103, wherein the computer program is configured to cause the computer to add the biometric data accessed in step a) for the patient to the database.

107. The computer program of claim 97, wherein the computer program is configured to cause the computer to compute a model of the patient's spine using the parameters.

108. The computer program of claim 86, wherein the computer program is configured to cause a computer to read data from an implant database in which data relating to all available components of the implant are stored.

109. The computer program of claim 107, wherein the computer is configured to cause the computer to access data, which have been obtained during an implant surgery as a result of measurements, but before an implant is implanted, and to enter these data into the spine model.

110. The computer program of claim 86, wherein the implant comprises a cage which is configured to be inserted into an intervertebral disk compartment.

111. The computer program of claim 97, wherein the computer program is configured to cause the computer to display a graph in which, for at least two adjacent vertebrae, a position of maximum inclination, a position of maximum reclination, and a neutral position are shown as symbols on a horizontal scaled line.

112. The computer program of claim 111, wherein the computer program is configured to cause the computer to display statistical data obtained from other persons for at least one of the positions indicated on the scaled line by symbols.

113. The computer program of claim 112, wherein the computer program is configured to cause the computer to display the statistical data as frequency distribution curve.

114. A computer program of claim 86, wherein the medium is a data carrier.

115. A computer program of claim 86, wherein the computer program is installed on a computer.

116. A computer program stored or running on a medium and configured to cause the computer to perform the following steps:
a) accessing biometric data which relate to the spine of a patient, the spine having at least one compromised spine segment;
b) accessing biometric data which relate to the spine of other persons;
c) comparing the biometric data accessed in step a) to the biometric data accessed in step b);
d) outputting a proposal for a configuration of an implant that is to be inserted into an intervertebral disc compartment of the compromised spine segment.

117. A computer program stored or running on a medium and configured to cause a computer to perform the following steps:
a) accessing biometric data which relate to a spine of a patient, the spine having at least one compromised spine segment, wherein the biometric data comprise image data obtained from images of the patient's spine in different spine positions, the images having been taken by using medical imaging techniques;
b) graphically displaying at least one of the images or a portion thereof, and
c) prompting a user to assign a first identifier to one of a plurality of vertebrae shown in the at least one image;
d) automatically recognizing the vertebrae shown in the at least one image using a pattern recognition algorithm;
e) automatically assigning different identifiers to other vertebrae displayed in the at least one image;
f) displaying the identifiers assigned in step iii) in the at least one image.
